SEQUENCE LISTING (116 sequences)

SEQ ID NO: 1 - DNA, 15962 bp, Artificial Sequence
  BPIV3 Backbone
gtcgactaatacgactcactatagggagattggtctgatgagtccgtgaggacgaaacggagtctagactccgtcaccaaacaaggggagagacttgtttggaaatatgaatttaaatagaaattaacttaggattaaagaactttaccgaaaggtaagggagaagaaatccttaaattgtcatcatgttgagtctgtttgatacattcagtgcacgcaggcaggaaaacataaccaaatcagctggtggagctgttattcctgggcaaaagaacacagtctctatatttgcccttggtccctcaataacagatgacaatgataaaatgacattagctctcctgttcttatctcactcactagataatgagaagcagcatgcacaaagagctggatttttagtctccttgctatccatggcttatgctaacccagaattatacctaacatcaaatggtaataatgcagatgtcaaatatgttatctatatgatagaaaaagactcagggagacaaaaatacggtggctttgttgttaagactagggaaatggtttatgagaagacaactgattggatgtttggaagtgatcttgagtatgatcaagataatatgttacagaatggtaggagcacctccacgattgaagatcttgttcacacatttgggtatccagcatgccttggagctcttataatccagatttggataatacttgttaaagctataaccagtatctcaggattgagaaaaggtttttttactcggttagaggcatttagacaggatgggacagtcaaatccagtctggtattgagtggagatgcagtagagcagatcggatcgatcatgaggtctcagcaaagtttggttacgcttatggtcgagacactgataacaatgaacacaggaaggaatgatctaacaacgatagagaagaacatacaaattgtaggaaactacataagagatgcaggccttgcttcattctttaatacaatcagatatggcattgaaaccagaatggcagctctaacattgtctactcttagaccagacatcaacagactcaaagcattaatagagttatacctatcaaaggggccacgtgccccttttatatgtattttaagggatcctattcatggcgagtttgcaccaggcaattatcctgctctctggagttatgcaatgggtgtagcagttgtccagaacaaagctatgcaacagtatgtaacaggacggtcctatctagatattgagatgttccaactaggtcaagcagtggcacgtgacgccgagtcacaaatgaattcaatattggatgaagaattggggattacacaagaggccaagcagagtttgaagaaacacatgaagaaaatcagtagttcggacacaactttctacaaacctacagggggaacagccatagaaatggcaatagacgaggagatagaacaaccagaatccagaggagaccaggatcaaactgatgatcttcagtcatctataatcccctatgcatgggcagacgaggcaggcaacaaaaaccaagcagggtcaaccgcagacgttgacagcctcggagttgagcaaagaaacatcagagatagattgaacaaaaggctcaacgaaaagagaagacagaatgaatcaaaatcagctgacataacaaataatgctgaccagactgagatagatgacctgttcagcgcattcggaagtagctaattaaatagtggtgattattaccatttgcaacaaataagaaaaacttaggattaaagagattacccagatttcacaagcacaatcaaaccagcacccagagacaacacggacagagcagaagaggcaccaggggaagaaccaacaaacaggaaagaggaaggaacaacagaaaacaggggcaaacaccaaagagcaaaggaacaccaccaacgaacataccagcaaccccacaacagaacacaagaaaaaagaatagaaaacgaagagcaccaaagaccacgcgtctcgagaccggaaatgtagcaccaaacatcaagggtataaaaaacttaggattaacggagattatccaatcctgacacagcagaccaagaccagactccatctacaacccaaccgatcgagaattcatggaagacaatgttcaaaacaatcaaatcatggattcttgggaagagagatcaggagacaagtcatccgacatctcatcagccctcgacatcattgaattcatactcagcaccgatccccaagaaagcgcggtcagcgacaatgagaacggcgcaggggtcacaaggctcagcacaacaatccaccggcccgacccgaaatcaacaaagacaggcgaggaaaatagtggaccaactaacgaagatcgacagcttgggacatcatacaaatataccgcagaaacaaaagatagagttgttaatcaggaagctgtacagagaagaaataggagaggaagcagcccagatagtagaactgagactatggtcattagaagaatcaccggaggcagctcagatcctgacaatggaaccgaaatccaggaaaatcttgattacaatgaaactagagaggtggataagaaccctactaaggggaaagtgcgacaacttgaaaatgttccagtcaaggtaccaggaagtgatgccatacctccagcaaaatcagatggagacagtgatgatggaggaggcctggaatctatcagtacacctaatccaagacatactagcctagtgaccacagcaacaccagatgacgaagaagagcttatctcgaaaaacaaaaggtcaaaaaagcaccagctaacaaaccagagaagtaacaaagaaattaaaaaagggggagaggaggagatttggaagcaagcagatatcgaccatcagtcatccagattggactacgaaccagacttcaaaggatcgaagaggaaccagaggatcctcaagaccagcacagatgcaggggaaccaacaagaatacagaatgggtcccaaaggaagagaatcacatcctggaacatcctcaacaacaagagcagcaatcgagaggaacagacagtcagaaatccccaacgatcagcacatggacagaagcaaacaatggtatcagacagatcagctccagaacaacaagtcaaagcacagaagacgaacggagaggaaggaaaggacacagaagagagcactcaatttacagaaagggcgattacattattacagagtcttggtgtaatccaatctgcagcaaaattagacctgtaccaagacaagagagttgtatgtgtggcgaatgtcctaaacaatgcagatactgcatcgaagatcgattttttagcgggtctgatgattggagtatcgatggatcaagacaacagacttaatcagattcaaaatgagataatagatttaaagactgatctcaaaaagatggatgaatcacacagaaggttgattgagaatcaaaaagaacaactatcattaatcacatcattaatttcgaatcttaagatcatgacagagagaggaggaaagaaagatcaggttgagacaagtggaagaacatccatgataaaaacaaaaatgaaagaagagaagataaggaaagttaagttcgatcctcttatggaaacacaaggcatcgaaaagaatgttcctgatttatacagatcgatagagaaggcaccagaaagtgacgtgcagatcaaatcagaaattagtaggttaagtgatgaatcgaatgcaactagactaataccaagaagaataagcagcacaatgaggtcattaatcataatcatcaataacagcagcctgtcatcaaaggcaaaacagtcatacataaatgaactcaagctctgcaagagtgatgaagaagtatctgaattgatggatatgtttaatgaagacattagctcccaatagacagccaaacgacaatcaccaccacagaacacactgacacacatcagcaaaccaaagactatccccaactaccaagtcaatcaacacaccacagtagttcccagacacaagccaatcaagaaaaacttaggacaaaagagccaatcagtccctcaacaaaatgagcatcacaaactccgcaatatatacatttccagaatcttcattttctgaaagtggcaatatagagccactaccactaaaggtcaacgaacaaaggaaggcaattcctcacatcagagttgttaaaataggtgacccacccaaacatggttcaagatatcttgatgtatttctactgggtttttttgagatggagcggtcaaaggacagatatgggagtataagtgatctggatgatgatccaagttataaagtttgtggttctggatctctaccacttgggctagcaagatacactgggaatgatcaggaactcctacaagccgcaattaagttagacatagaagtgagaagaaccgtcaaagccacggagatgatagtctacaccgtacagaacattaaacctgaactgtacccatggtcaagcaggttaagaaaagggatgttatttgacgccaacaaagttgcgcttgcacctcaatgtcttccactcgatagagggataaaattcagagtaatatttgtgaattgtacagcaattggatcaataacactctttaaaattcccaaatctatggctttgctatcattgccaaacactatatcaataaatttacaggtacacataaaaacaggaattcaaactgactctaagggtgttgtccagatcttagatgaaaaaggtgagaaatcgttaaactttatggtccatcttggattaattaaacggaaggtaggtcgaatgtattcagtagagtattgcaaacaaaagattgagaagatgagactactattttcattgggattagttggaggaatcagctttcatgtcaatgcaactgggtccatttcaaaaacgttggcgagtcaactagcgttcaaaagggaaatctgttatcctctcatggatttaaatccacacctaaatctggttatatgggcatcatcaattgaaattacaagagtggatgcaatccttcaaccttcattgcccggtgagttcagatattacccaaacataatagcaaaaggagtcgggaaaatcaagcagtaacaccgacagcctggacaagcaacattataaatcagatcatttaaaaaaccaaaatcaaaaacttaggattaaagagattacccagatttcacaagcacaatcaaaccagcacccagagacaacacggacagagcagaagaggcaccaggggaagaaccaacaaacaggaaagaggaaggaacaacagaaaacaggggcaaacaccaaagagcaaaggaacaccaccaacgaacataccagcaaccccacaacagaacacaagaaaaaagaatagaaaacgaagagcaccaaagatgaccaccatagttgcaacaacagttatattattattacctttatcactatgtcagatagatataacaaagttgcaacgagtaggggtgctagtcaacaatccaaaaggcatgaagatttcacaaaattttgaaacaagatacttaatattaagtttaataccaaagatagagaactcacaatcatgtggagaccaacaaataaatcaatataagaaattattagatagattaattattcctttatatgatgggttaaaattacagaaagatgtaatcgtagtaagacatgagtctaataacagtactagttccaggaagaagcggttcttcggagaaataattgggacaattgcgatagggatagccacatcagctcaaataactgcagcagttgcccttgttgaagccaaacaggcaagagcagatatagacaagctcaaggaagccatcagagatacaaacaaggcagtacagtcaattcaaagctcagtaggaaatctaattgttgcggttaagtcagttcaagattacgtcaataatgagattgtaccatcaatagcaagattaggctgtgaagcagcaggattacaactcggaattgcattgacacaacattactcagagttaacaaatatatttggtgataacataggaacactgaaggagaagggaataaaattacaagggatagcatcattatatcgcacaaacataacagagatattcactacttcaacagttgatcaatatgacatttatgatctattgttcactgagtcaatcaaaatgagagttatagatgtagacttaaatgactattcaattacactacaagttagactacctttattaacaaaattatcaaacactcagatttatagagtggattctatatcatataatatccaaggtaaggaatggtacattcctcttcccaatcacatcatgacaaagggggcatttttgggtggtgctgatataaaagagtgtatagaggcattcagcagttacatatgtccctctgacccaggttacatattaaatcgtgagatagaaaactgtctatcagggaatatcacacagtgtccgaaaaccgttgttacatcagacatagtaccaagatatgcatttgttaatggcggagtaattgccaattgtataccaaccacttgtacatgtgatgggattgacaataggatcaaccaggcacccgatcaagggataagggtaataacacacaaagaatgtcaagtgataggtataaacgggatgttattcagacctaacaaagaagggactttggcaacttatacatatgatgacattgttttaaacaattctgtcgcgcttgacccaattgacatatcaatggaactcaacaaagctaaattggaattagaagaatcaaaagaatggataaagaagtcaaatcaaaaattggactctgttggaggttggtatcagtcaaatataacaattactataataatagtgatgataatgattctgtttataatcaacatgataatcattataataatctttaaacaattcaagattcgcaatagacacccaattgacaagagcaatgaaccctatatactgactaggaaacagtagggaaatgtagcaccaaacatcaagggtataaaaaacttaggaacaaagcagtcccactcagagacaaagaacagacgacacggaatcccaacctcaaaaatggaatattggaggcacacaaacaacgccaaaaacaccaacactgaattccaaacagagacaactaggcgtaataataaagttacaaataccatgatgtccaccttcggggcaatactctcaactatattgttgacagtgttcataatgatactggtaggtttgattcaagaaagcaaccacaacaaaatagcatcacaacaaatgagaagagaattcgcagaaattgagaaaaaaatccagcaagctgcagatgaaattggtacatcaatacaatcgggaataaacacaagactcctaactatccagagtcatgtccaaaattatattccattatctttgacacaacaaatgtcggatcttagaaaatttatcaatgaattagcaaataagagagatcaacaggaggtaccaatacagagaatgactcatgacagcgggattgaacccctaaaccctgacaaattctggagatgtacctctggcaatccatccttgacaagtagcccaaagataagactaataccaggtcctagtctattagcagcatctaccactgtaaatggttgtataaggatcccgtccttcgtaatcaaccatctaatatatgcttacacttccaaccttattgttcaaggttgccaggatatagggaagtcttatcaggtattacaaatcgggatcatcactataaattcagacctggtacctgacttaaaccctagagttacacatacttttaatattgatgataatagaaagtcctgttctttggcactactaaatacagatgtatatcagttgtgctcaactccgaaagttgacgaaagatcagactatgcatcaacagggattgaagacattgtacttgatattattactaacaatggattaatcatcacaacaagatttaccaatgataatataacatttgacaaaccatatgcagcactgtacccatcagtaggaccggggatttattataaaggtaaggttatttttctcggatatggtggattagaacatgcagagaatggtgatgtaatatgcaatttaactggttgtccaggcaaaacacagagagactgtaatcaagcttcctacagcccatggttttcagataggagaatggtgaactctattattgttgttaataaaggtgtggacgcaactttcaatttaagagtatggactatcccaatgagccaaaactattggggatcagaagggagattacttttattaggcaataagatatacatatatactagatctacaagttggcatagtaaactccaattagggacaattgatattaccaattacagtgatatcaggatcaattggacctggcatgatgcattatcgagaccaggaaatgatgattgtccgtggggtcactcatgcccggatgggtgtataacaggggtttatactgatgcatacccgttaaacccatcaggaagtgttgtgtcatcagttattcttgattctcgaaaatccagggaaaatccgatcatcacttacgcaacagacacacgtagaataaatgaactggctatatataacagaacacttccagctgcatacacaacaacaaattgcattatgcattatgataagggatactgcttccacattgtcgagataaatcataggagcctaaatacattccaacctatgttattcaaaacagaaattccaaagaactgtagttgagttgactctcccatgttaattatcaaacacggaagacaagcatcaacaggtaggggatccaaaaaatgagatgacaaatagaggaaaaataaaaaacttaggagaaaagtttgtcgggaaaatggacaccgaattcagcggtggcacaacatctgatattttatatccagaatgtcatctcaattctcctatagtgaaagggaaaatagcacaattacacactataatgagtttgcctcaaccttatgatatggatgatgactcaatactagttataactaggcagaagattaggctgaataaattagataaaagacaacgatcaatcaggaaattgaaatctgtcctaatggaaagagtaattgacttaggtaaatataactttattaggtatccagaaatgtctagtgaaatgttccaattgtgtatacctggaattaatagcagaataaatgaattattaagtaaagcacataggacttataatcaaatgactgatggattaagggatttatgggttactatactatcaaaattggcatcaaaaaatgatggaagtaattataatattaacgaagatatcagtaacatatctagtgttcacatgacctatcaatcagatagatggtataatccattcaagacatggtttaccattaaatacgatatgaggaggctacaaaaagccaaaaatgaggttacatgtgataagtacaaggattacaatttattagaagatcaaaggaatgtattactgatacatacggagcttgtcctcatactagacaaacaaaattataaaggatatataataacccctgaattggtattaatgtattgtgacgtagttgaaggaagatggaacattagctcgtgtgcaaaactagaccctaaattacattcaatgtattacaaaggtaacaacttgtgggaaacaatagatgggttgttcccagaattaggagagaaaacttttgatataatatcattgttagaaccactagctttgtcactcatacaaactcatgatccagttaagcaattacgtggagcctttttgaatcatgtattatctgaaatggagtcaatattagacaccaggtacaagaccgaagaaacacctgaattggactacatagacaaaattctagatgtattcaaagaatcaacaatagatgaaatagcagagattttctcttttttccgaacttttggacatcctccattagaagctagtatagcagcagaaaaagtcaggaaatatatgtatactgagaaaagcctaaattttgatactattaataaatgtcatgctattttttgtacaatcattataaatgggtatagagaaagacacggaggacaatggcctccaattatattgcctgctcatgcacatgaattcattataaatgcttatggatcaaattctgctatatcatacgaaaatgctgttgattactataggagctttataggaattaagttcaacaaatttatagaacctcaattagatgaagacttaactatttatatgaaagataaagcattatctccaaaaaaatcaaactgggacaccgtatatccatcctcaaacttattgtatcatactagtacatcccatgattcaagaaggttagttgaggtttttatagcagatagtaaatttgaccctcatcaggtattagattatgtggagtcaggagaatggttgaatgatcctgagttcaatatctcatatagtctaaaagagaaggagataaagcaagaaggtaggctttttgcaaagatgacatataaaatgagagctgcgcaagttttgtcagaaacgttattagcaaacaatatagggaaattctttcaggagaatggaatggtcaaaggagagattgagctacttaaaagattgacaacaatatctatgtctggagtcccacgatacaatgaagtatacaataatcccaaaagtcatacagatgagatcaaagcttacaacgcgattagtaattctaatttatcttctaaccaaaaatcaagaagattcgaattcaagtcaacagacatatacaatgacggatatgaaactgtaagttgttttttaacaacagatctaaagaaatattgtttgaattggagatatgaatcaacagctctatttggtgatacttgcaatcagattttcgggttaaaaaagttattcaattggttgcatcctcgccttgaaaagagtacaatatatgttggagatccctattgcccgcccttagatatagaacacttacaacttgatgatcaccctgattcagggttttacgttcataatcctagaggaggaatagaaggattttgtcaaaaattgtggacacttatatctattagcgcaatacacctagcggctgtcaaaataggtgttagagtgactgcaatggtgcaaggagacaatcaagccattgctgtgactacaagggtgccaaataactatgattacaggatcaagaaggaaattgtttacaaggatgtggttaagttttttgattctttaagagaggttatggatgatctgggtcatgaacttaaattaaatgaaacaataataagcagtaaaatgtttatatacagcaaaagaatatattatgatggaagaattctccctcaagctttaaaagcattatctagatgtgtcttttggtctgaaaccattatagatgaaacaagatcggcatcttctaatttagcaacatcatttgcaaaagctattgagaatggttattcacctgtgttgggatatgtgtgttcaatattcaaaaatatccaacaattgtatatagcactcggaatgaatattaatcctactataactcagaatatcagagatcaatattttaggaacacccattggatgcaatatgcttctttaatacctgcaagtgttggagggtttaattatatggccatgtctaggtgttttgtaagaaacattggggaccccacggttgccgcattggcggatattaaaagatttataaaagcaaatctgttggatcgtggtgttctttatcgaattatgaatcaagaaccaggtgaatcttcgtttctagactgggcttctgatccgtactcatgtaacttaccacaatcccaaaatataacaactatgattaaaaatataactgcaagaaatgtactacaagattcaccaaatccattattatcaggattatttacaagcacgatgatagaagaggatgaagaactcgctgagtttttgatggataggagaataattcttccaagagttgcacatgatattttagataattctcttactggaatcagaaatgctatagcaggaatgctggacacaacaaaatcgttaattcgagtagggataaatcgaggagggttaacttatagtttgttaagaaaggtaagtaactatgatcttgtacaatatgagacacttagtagaaccttaagattaattgttagtgataagataaagtatgaagacatgtgctcggtagatctagctatctcgttaagacaaaaaatgtggatgcatttatcaggaggaagaatgataaatgggcttgaaacacctgatccattagaactattgtctggagtaataataacagggtctgagcactgcaggatatgttattcaactgaaggagaaagcccatatacttggatgtatttacctggtaatctgagtataggatcagccgaaaaagggattgcatcattaagagtcccttattttgggtcggtaacagatgagagatctgaggcacaactggggtacattaagaatttaagcaaaccagcaagagctgctataagaatagcaatgatctatacttgggcatttggcaatgatgagatatcctggatggaggcatcacagattgcacagacacgtgcaaatttcacgttagatagcttaaagattctgacaccagtgactacatcaacaaatttatcacacagattaaaggataccgctacccagatgaaattttctagtacatcacttatcagagtaagcagatttatcacaatatcgaatgataatatgtctatcaaagaagctaacgaaacaaaagatacaaatcttatttatcaacaggtaatgttaacagggttaagtatatttgaatatttatttagattagaagaaaatacagggcacaatcctatggttatgcatctacatatagaagacgggtgttgtattaaagaaagttacaatgatgatcatatcaatccagagtccacattggagttgattaggtatcctgaaagtaatgagtttatatatgacaatgaccctctcagagatgtagacctgacaaaactaatgattataagagatcactcctatactattgatatgaattattgggatgacacagatattatacacgcagtatcaatatgtacagcaatcacaatagcagacaccatgtcacagcttgaccgggacaatcttaaggagctaatagttattgcaaatgatgatgatataaatagtcttatcactgaattcctaacccttgacatattagtatttcttaaaacattcggaggattacttgttaatcaattcgcatatactctatatgggttgaaaatagaaggaagggatcctatctgggactatataatgaggacactaaaagacacctcacactcaatattaaaagttttgtcaaatgcattatcccatccaaaggtttttaaaaggttctgggattatggagttttaaatcctatctatgggcctaacactgctagtcaagaccaaatcaagcttgctctttctgtttgtgagtattctcttgatttatttatgagagaatggctgaatggtgcatcgcttgaaatctatatttgcgacagcgatatggaaatagcaaatgacaggagacaagcatttgtttcgagacatctcgcgtttgtgtgttgtttagcagaaatagcatcatttggaccgaatttattaaacttgacgtatttggaaaggttagaagaattaaagcggtatttagatttaaacattaaagaagatccaactcttaaatacgtacaagtatcaggactattaatcaaatcattcccttcaactgttacatatgtaagaaagacagcaattaaatatctaagaattcgtggaattgacccacctaacatacttgaggattgggatcccatagaagatgagaatatattagataatattgttaggactgtgaatgataattgtaacgataatccaaggaaatgtaagagtaattatttttggggtttagctcttaaaaattatcaagttgtgaggataagatctataaccagtgatcctgaagtttatgagacctcagatgatattgaacatggattgacacttcctgagggaggaagttatttatcacaccagctgcgattatttggagtgaatagtacaagttgtatcaaagcccttgagctatcgcagattctaatgaaagaagttaaaaaagataaggatagacttttcctaggagagggagcaggtgctatgctagcatgttatgatgccacacttggtcccgcaataaattattacaattctggtctaaatattacagatgtaattggtcaaagagaattaaaaatctttccatcagaagtttcattagtaggtaagaaactaggtaatgtgacacagatcctcaatcgtgtgaggatattatttaacgggaaccctaactcaacatggataggaaatatggaatgcgaaagtttaatatggagtgaattacatgacaaatcaattggtttagtacattgtgatatggaaggagcaataggtaaatcagaggaaactgtcctacatgaacactatagtattattagaatcacatatttagttggagacgatgatgttatcttggtatccaaaatcataccaaccatcactccaaattggtctaaaatacttcatttatataaattgtattggaaggatgtgagaataatatcccttaagacatccaaccccgcctcgacagagctgtatctaatttctaaggaagcatattgtactgtgatggagcctagtaatcttgtgttatcaaaactcagaagaatatcatcaatagaagagaataatctactaaagtggataatcttatctaaaaagaagaataatgaatggttgcagcatgaaattaaagaaggagaaagagactatggagtaatgagaccctatcacacagcactgcaaatattcggattccagattaatctaaaccatctagcaaaagagtttctatcaactcctgatttaaccaatattaacattgtgatacaaagtttcataagaacagtcaaagatgttatattcgaatgggtaaacataactcatgacaacaaaagacataaattaggagggaaatataatttgtttccgctcaaaaatagaggaaagttaaaattactttcacggagattagtgttgagctggatatcattatctctatcgaccagattactgacgggtcgtttcccggatgagaagtttgagggtagagcacagaccgggtttgtgtcattagctgacactgatctcgaacctctaaaattactatccaaaactcttgtcagagattataaagaacacataggattgatatcatattggtttctaaccaaagagatcaaaatactgatgaaacttataggaggagttaaattattggggatccctaaacaatataaagaacttgaacaccaatcatatccagattatgaatatgataatgaatttgatattgattaatgtataaatccagtaaatagtgtacttaatccctatctgtatatattaccaaccaaaaacaataagaaaaacatatgatatatatataccaaacagagtttttctcttgtttggtggccggcatggtcccagcctcctcgctggcgccggctgggcaacattccgaggggaccgtcccctcggtaatggcgaatgggacgctagttattgctcagcgggctagttattgctcagcgggtcgac

SEQ ID NO: 2 - protein, 540 aa, Artificial Sequence
  Mosaic BPIV3 Fusion Polypeptide
  misc_feature (6)..(6): Xaa can be any naturally occurring amino acid
  misc_feature (8)..(9): Xaa can be any naturally occurring amino acid
  misc_feature (13)..(13): Xaa can be any naturally occurring amino acid
  misc_feature (96)..(96): Xaa can be any naturally occurring amino acid
  misc_feature (100)..(100): Xaa can be any naturally occurring amino acid
  misc_feature (104)..(104): Xaa can be any naturally occurring amino acid
  misc_feature (428)..(428): Xaa can be any naturally occurring amino acid
  misc_feature (431)..(431): Xaa can be any naturally occurring amino acid
  misc_feature (444)..(444): Xaa can be any naturally occurring amino acid
  misc_feature (486)..(486): Xaa can be any naturally occurring amino acid
  misc_feature (502)..(502): Xaa can be any naturally occurring amino acid
  misc_feature (509)..(509): Xaa can be any naturally occurring amino acid
  misc_feature (513)..(513): Xaa can be any naturally occurring amino acid
  misc_feature (518)..(519): Xaa can be any naturally occurring amino acid
  misc_feature (527)..(527): Xaa can be any naturally occurring amino acid
  misc_feature (530)..(531): Xaa can be any naturally occurring amino acid
  misc_feature (538)..(538): Xaa can be any naturally occurring amino acid
MIITVXTXXILLXPLSLCQIDITKLQRVGVLVNNPKGMKISQNFETRYLILSLIPKIENSHSCGDQQINQYKKLLDRLIIPLYDGLKLQKDVIVVXHESXNNTXSRTKRFFGEIIGTIAIGIATSAQITAAVALVEAKQARSDIDKLKEAIRDTNKAVQSIQSSVGNLIVAVKSVQDYVNNEIVPSITRLGCEAAGLQLGIALTQHYSELTNIFGDNIGTLKEKGIKLQGIASLYRTNITEIFTTSTVDQYDIYDLLFTESIKMRVIDVDLSDYSITLQVRLPLLTKLSNTQIYKVDSISYNIQGKEWYIPLPNHIMTKGAFLGGADIKECIEAFSSYICPSDPGFILNHEIENCLSGNITQCPKTVVTSDIVPRYAFVNGGVIANCIPTTCTCNGIDNRINQSPDQGIKIITHKECQVIGINGMLFXTNXEGTLATYTFDDIXLNNSVALNPIDISMELNKAKLELEESKEWIKKSNQKLDSVGXWYQSSATITIIIVMIXILFIINXIIIXIIIKXXRIRNRHPXDKXXEPYVLTXRQ

SEQ ID NO: 3 - protein, 572 aa, Artificial Sequence
  BPI3V Hemagglutinin-Neuraminidase [HN] mosaic polypeptide
MEYWRHTNNAKNTNTEFQTETTRRNNKVTNIMMSIFGAILSTILLTVFIMILVGLIQESNHNKIASQQMRREFAEIEKKIQQAADEIGTSIQSGINTRLLTIQSHVQNYIPLSLTQQMSDLRKFINELANKRDQQEVPIQRMTHDSGIEPLNPDKFWRCTSGNPSLTSNPKIRLIPGPSLLAASTTVNGCIRIPSFVINNLIYAYTSNLIVQGCQDIGKSYQVLQIGIITINSDLVPDLNPRVTHTFNIDDNRKSCSLALLNTDVYQLCSTPKVDERSDYASTGIEDIVLDIITNNGLIITTRFTNDNITFDKPYAALYPSVGPGIYYKGKVIFLGYGGLEHAENGDVICNLTGCPGKTQRDCNQASYSPWFSDRRMVNSIIVVNKGVDTTFNLRVWTIPMRQNYWGSEGRLLLLGNKIYIYTRSTSWHSKLQLGTIDITNYSDIRINWTWHDALSRPGNDDCPWGHSCPDGCITGVYTDAYPLNPSGSVVSSVILDSRKSRENPIITYATDTRRVNELAIYNRTLPAAYTTTNCIMHYDKGYCFHIVEINHRSLNTFQPMLFKTEIPKNCS

SEQ ID NO: 4 - DNA, 111 bp, Artificial Sequence
  Primer
gcgtcgactaatacgactcactatagggagaaacatctgatgagtccgtgaggacgaaacggagtctagactccgtcatgttgagtctgtttgatacattcagtgcacgca

SEQ ID NO: 5 - DNA, 38 bp, Artificial Sequence
  Primer
gcaagcttttagctacttccgaatgcgctgaacaggtc

SEQ ID NO: 6 - DNA, 38 bp, Artificial Sequence; identical to SEQ ID NO: 5
  Primer

SEQ ID NO: 7 - DNA, 60 bp, Artificial Sequence
  Primer
gcaagcttctattgggagctaatgtcttcattaaacatatccatcaattcagatacttct

SEQ ID NO: 8 - DNA, 101 bp, Artificial Sequence
  Primer
gccccgggtaatacgactcactatagggagatccatctgatgagtccgtgaggacgaaacggagtctagactccgtcatggacaccgaattcagcggtggc

SEQ ID NO: 9 - DNA, 60 bp, Artificial Sequence
  Primer
gcaagcttttaatcaatatcaaattcattatcatattcataatctggatatgattggtgt

SEQ ID NO: 10 - protein, 39 aa, Bovine parainfluenza virus 3
MEDNVQNNQIMDSWEERSGDKSSDISSALDIIEFILSTD

SEQ ID NO: 11 - protein, 39 aa, Bovine parainfluenza virus 3
MEDNAQNNQIMDSWEERSGDKSSDISSALDIIEFILSTD

SEQ ID NO: 12 - protein, 39 aa, Bovine parainfluenza virus 3; identical to SEQ ID NO: 10

SEQ ID NO: 13 - protein, 39 aa, Bovine parainfluenza virus 3; identical to SEQ ID NO: 10

SEQ ID NO: 14 - protein, 39 aa, Bovine parainfluenza virus 3; identical to SEQ ID NO: 10

SEQ ID NO: 15 - protein, 39 aa, Bovine parainfluenza virus 3; identical to SEQ ID NO: 10

SEQ ID NO: 16 - protein, 39 aa, Bovine parainfluenza virus 3
NQEAIQRRNRRGSSSDSRTETMVIRRITGGSSDPDNGTE

SEQ ID NO: 17 - protein, 39 aa, Bovine parainfluenza virus 3
NQEAIQGRNRRGSSSDSRTETLVIRRITGGSSDPDNGTE

SEQ ID NO: 18 - protein, 39 aa, Bovine parainfluenza virus 3
NQEAIQGRNRRGSSSDSRTETMVIRRITGGSSDPDNGTE

SEQ ID NO: 19 - protein, 39 aa, Bovine parainfluenza virus 3; identical to SEQ ID NO: 18

SEQ ID NO: 20 - protein, 39 aa, Bovine parainfluenza virus 3; identical to SEQ ID NO: 18

SEQ ID NO: 21 - protein, 39 aa, Bovine parainfluenza virus 3; identical to SEQ ID NO: 18

SEQ ID NO: 22 - protein, 39 aa, Bovine parainfluenza virus 3
RHTSLVTTATPDDEEELISKNKRSKRHQLTNQRDNKEIK

SEQ ID NO: 23 - protein, 39 aa, Bovine parainfluenza virus 3
RHTSLVTTATPDDEEELILKNKRSKRHQLTNQRDNKEIK

SEQ ID NO: 24 - protein, 39 aa, Bovine parainfluenza virus 3; identical to SEQ ID NO: 22

SEQ ID NO: 25 - protein, 39 aa, Bovine parainfluenza virus 3; identical to SEQ ID NO: 22

SEQ ID NO: 26 - protein, 39 aa, Bovine parainfluenza virus 3; identical to SEQ ID NO: 22

SEQ ID NO: 27 - protein, 39 aa, Bovine parainfluenza virus 3; identical to SEQ ID NO: 22

SEQ ID NO: 28 - protein, 39 aa, Bovine parainfluenza virus 3
ILNNKSSNREEQTVRNPQRSAHGQKQTMVSDRSAPEQQV

SEQ ID NO: 29 - protein, 39 aa, Bovine parainfluenza virus 3
ILNNKSNNREEQTVRNPQRSAYGQKQTMVSDRSAPEQPV

SEQ ID NO: 30 - protein, 39 aa, Bovine parainfluenza virus 3
ILNNKSSNREEQTVRNPQRSAYGQKQTMVSDRSAPEQQV

SEQ ID NO: 31 - protein, 39 aa, Bovine parainfluenza virus 3; identical to SEQ ID NO: 28

SEQ ID NO: 32 - protein, 39 aa, Bovine parainfluenza virus 3; identical to SEQ ID NO: 28

SEQ ID NO: 33 - protein, 39 aa, Bovine parainfluenza virus 3; identical to SEQ ID NO: 28

SEQ ID NO: 34 - DNA, 28 bp, Bovine parainfluenza virus 3
acttcaaaggatcgaagaggaaccagag

SEQ ID NO: 35 - DNA, 28 bp, Bovine parainfluenza virus 3
acttcaaaggatcgaagaggagccagag

SEQ ID NO: 36 - DNA, 28 bp, Bovine parainfluenza virus 3
acttcaaaggatcgaagaggaaccagag

SEQ ID NO: 37 - DNA, 28 bp, Bovine parainfluenza virus 3
acttcaaaggatcgaagaggaaccagag

SEQ ID NO: 38 - DNA, 28 bp, Bovine parainfluenza virus 3
acttcaaaggatcgaagaggaaccagag

SEQ ID NO: 39 - DNA, 28 bp, Bovine parainfluenza virus 3
acttcaaaggatcgaagaggaaccagag

SEQ ID NO: 40 - DNA, 28 bp, Bovine parainfluenza virus 3
catcctggaacatcctcaacaacaagag

SEQ ID NO: 41 - DNA, 28 bp, Bovine parainfluenza virus 3
catcctggaacatcctcaacaacaagaa

SEQ ID NO: 42 - DNA, 28 bp, Bovine parainfluenza virus 3
catcctggaacatcctcaacaacaagag

SEQ ID NO: 43 - DNA, 28 bp, Bovine parainfluenza virus 3
catcctggaacatcctcaacaacaagag

SEQ ID NO: 44 - DNA, 28 bp, Bovine parainfluenza virus 3
catcctggaacatcctcaacaacaagag

SEQ ID NO: 45 - DNA, 28 bp, Bovine parainfluenza virus 3
catcctggaacatcctcaacaacaagag

SEQ ID NO: 46 - DNA, 28 bp, Bovine parainfluenza virus 3
cagacagatcagctccagaacaacaagt

SEQ ID NO: 47 - DNA, 28 bp, Bovine parainfluenza virus 3
cagacagatcagctcccgaaccacaagt

SEQ ID NO: 48 - DNA, 28 bp, Bovine parainfluenza virus 3
cagacagatcagctcccgaacaacaagt

SEQ ID NO: 49 - DNA, 28 bp, Bovine parainfluenza virus 3
cagacagatcagctcccgaacaacaagt

SEQ ID NO: 50 - DNA, 28 bp, Bovine parainfluenza virus 3
cagacagatcagctcccgaacaacaagt

SEQ ID NO: 51 - DNA, 28 bp, Bovine parainfluenza virus 3
cagacagatcagctcccgaacaacaagt

SEQ ID NO: 52 - DNA, 28 bp, Bovine parainfluenza virus 3
ttacattattacagagtcttggtgtaat

SEQ ID NO: 53 - DNA, 28 bp, Bovine parainfluenza virus 3
ttacattattacagaatcttggtgtaat

SEQ ID NO: 54 - DNA, 28 bp, Bovine parainfluenza virus 3
ttacattattacagagtcttggtgtaat

SEQ ID NO: 55 - DNA, 28 bp, Bovine parainfluenza virus 3
ttacattattacagagtcttggtgtaat

SEQ ID NO: 56 - DNA, 28 bp, Bovine parainfluenza virus 3
ttacattattacagagtcttggtgtaat

SEQ ID NO: 57 - DNA, 28 bp, Bovine parainfluenza virus 3
ttacattattacagagtcttggtgtaat

SEQ ID NO: 58 - protein, 40 aa, Bovine parainfluenza virus 3
NQEAIQGRDRRGGNSDSRTETMVTRRISRSSSDPNNGAQI

SEQ ID NO: 59 - protein, 40 aa, Bovine parainfluenza virus 3
NQETIQGGNRGRSSSDSRAEIMVTRGISRSSPDPNNGTQI

SEQ ID NO: 60 - protein, 40 aa, Bovine parainfluenza virus 3
NQKTVQGGYRRGSSPDSRTETMVTRGISRSSPDPNNGTQI

SEQ ID NO: 61 - protein, 40 aa, Bovine parainfluenza virus 3
NQETIQGGNRGGSSSDSRAETMVTRGISRSSPDPNNGTQI

SEQ ID NO: 62 - protein, 40 aa, Bovine parainfluenza virus 3
NQEAIQGRDRRGGNSDSRTETMVTGRISRSSSDPNNGAQI

SEQ ID NO: 63 - protein, 40 aa, Bovine parainfluenza virus 3; identical to SEQ ID NO: 59

SEQ ID NO: 64 - protein, 40 aa, Bovine parainfluenza virus 3; identical to SEQ ID NO: 59

SEQ ID NO: 65 - protein, 41 aa, Bovine parainfluenza virus 3
NQETVQGGYRRGSSPDSRTETMVTRRISRSSPPDPNNGTQI

SEQ ID NO: 66 - protein, 40 aa, Bovine parainfluenza virus 3
NQEAIQRRNRRGSSSDSRTETMVIRRITGGSSDPDNGTEI

SEQ ID NO: 67 - protein, 40 aa, Bovine parainfluenza virus 3
NQEAIQGRNRRGSSSDSRTETLVIRRITGGSSDPDNGTEI

SEQ ID NO: 68 - protein, 40 aa, Bovine parainfluenza virus 3
NQEAIQGRNRRGSSSDSRTETMVIRRITRGSSDPDNGTEI

SEQ ID NO: 69 - protein, 40 aa, Bovine parainfluenza virus 3
NQEAIPGRNRRGSSSDSRTETMVIRRITGGSSDPDNGTEI

SEQ ID NO: 70 - protein, 40 aa, Bovine parainfluenza virus 3; identical to SEQ ID NO: 68

SEQ ID NO: 71 - protein, 40 aa, Bovine parainfluenza virus 3
NQEAIQGRNRRGSSSDSRTETMVIRRITGGSSDPDNGTEI

SEQ ID NO: 72 - protein, 40 aa, Bovine parainfluenza virus 3
GYTSIVTAATPDDEEELLMRNNRPRRHQSTPQGDNKGIKK

SEQ ID NO: 73 - protein, 41 aa, Bovine parainfluenza virus 3
GYTSIVTAATLDDEEELLMKNTRPKRYQSTPQEEDDKGIKK

SEQ ID NO: 74 - protein, 40 aa, Bovine parainfluenza virus 3
GYTSIVTAATLDDEEELLMKNNRPRKYQSTPQNSDKGIKK

SEQ ID NO: 75 - protein, 40 aa, Bovine parainfluenza virus 3
GYTSIVTAATLDDEEELLMKNTRPRRYQSTPQEDDKGIKK

SEQ ID NO: 76 - protein, 40 aa, Bovine parainfluenza virus 3; identical to SEQ ID NO: 72

SEQ ID NO: 77 - protein, 40 aa, Bovine parainfluenza virus 3
GYTSIVTAATLDDEEELLMKNTRPKRYQSTPQEDDKGIKK

SEQ ID NO: 78 - protein, 41 aa, Bovine parainfluenza virus 3
GYTSIVTAATLDDDEEELLMKNTRPKRYQSTPQEDDKGIKK

SEQ ID NO: 79 - protein, 41 aa, Bovine parainfluenza virus 3
GYTSIVTAATLLDDEEELLMKNNRPRKYQSTPQNSDKGIKK

SEQ ID NO: 80 - protein, 40 aa, Bovine parainfluenza virus 3
RHTSLVTTATPDDEEELISKNKRSKRHQLTNQRDNKEIKK

SEQ ID NO: 81 - protein, 40 aa, Bovine parainfluenza virus 3
RHTSLVTTATPDDEEELILKNKRSKRHQLTNQRDNKEIKK

SEQ ID NO: 82 - protein, 40 aa, Bovine parainfluenza virus 3
RHTSLVTTATPDDEEELLSKNKRSKRYQLTNQRDNKEIKK

SEQ ID NO: 83 - protein, 40 aa, Bovine parainfluenza virus 3
RHTSLVTTATPNVEEELTSKNKRSKRHQLTNQRDNKEIKK

SEQ ID NO: 84 - protein, 40 aa, Bovine parainfluenza virus 3
RHTSLVTTATPDDEEELLSKNKRSKRHQLTNQRDNKEIKK

SEQ ID NO: 85 - protein, 40 aa, Bovine parainfluenza virus 3
RHTSLVTTATPDDEEELFSKNKRSKRHQLTTQRDNKEIKK

SEQ ID NO: 86 - DNA, 59 bp, Bovine parainfluenza virus 3
caaaacaacagaaacaagcaaggaaaatagtggaccagctaacaaaaatcgacagtttg

SEQ ID NO: 87 - DNA, 59 bp, Bovine parainfluenza virus 3
caaaacaacagaaacaagcaaggaaaatagtggatcagctaacaaaaatcgacagtttg

SEQ ID NO: 88 - DNA, 59 bp, Bovine parainfluenza virus 3; identical to SEQ ID NO: 86

SEQ ID NO: 89 - DNA, 59 bp, Bovine parainfluenza virus 3
agatagaaatgttaatcaggagactgtacagggaggatataggagaggaagcagcccag

SEQ ID NO: 90 - DNA, 59 bp, Bovine parainfluenza virus 3
agatagaaatgttaatcaggagactatacagggaggatataggagaggaagcagctcag

SEQ ID NO: 91 - DNA, 59 bp, Bovine parainfluenza virus 3; identical to SEQ ID NO: 89

SEQ ID NO: 92 - DNA, 59 bp, Bovine parainfluenza virus 3
aatctccagaagcagcccagatcctaacaatggaacccaaatccaggaagatattgatt

SEQ ID NO: 93 - DNA, 59 bp, Bovine parainfluenza virus 3; identical to SEQ ID NO: 92

SEQ ID NO: 94 - DNA, 59 bp, Bovine parainfluenza virus 3; identical to SEQ ID NO: 92

SEQ ID NO: 95 - DNA, 59 bp, Bovine parainfluenza virus 3; identical to SEQ ID NO: 92

SEQ ID NO: 96 - DNA, 59 bp, Bovine parainfluenza virus 3
aatctccagaagcagcccagatcctaacaatggaacccaaatccaagaagatattgatt

SEQ ID NO: 97 - DNA, 59 bp, Bovine parainfluenza virus 3; identical to SEQ ID NO: 92

SEQ ID NO: 98 - DNA, 59 bp, Bovine parainfluenza virus 3
tgatgatggaagaggcctggaatctatcagtacatttgattcaggatataccagtatag

SEQ ID NO: 99 - DNA, 59 bp, Bovine parainfluenza virus 3; identical to SEQ ID NO: 98

SEQ ID NO: 100 - DNA, 59 bp, Bovine parainfluenza virus 3; identical to SEQ ID NO: 98

SEQ ID NO: 101 - DNA, 75 bp, Bovine parainfluenza virus 3
tggtatgttggatacaacaaaatcactaattcgagtagggataagcagaggaggattaacctataacttattaag

SEQ ID NO: 102 - DNA, 74 bp, Bovine parainfluenza virus 3
aggaatgctggacacaacaaaatcgttaatcgagtagggataaatcgaggagggttaacttatagtttgttaag

SEQ ID NO: 103 - DNA, 74 bp, Bovine parainfluenza virus 3; identical to SEQ ID NO: 102

SEQ ID NO: 104 - DNA, 74 bp, Bovine parainfluenza virus 3; identical to SEQ ID NO: 102

SEQ ID NO: 105 - DNA, 74 bp, Bovine parainfluenza virus 3
aaagataagcaactatgatcttgtacaatatgagacacttagtaaaactttaagactaatagtcagtgacaaga

SEQ ID NO: 106 - DNA, 75 bp, Bovine parainfluenza virus 3
aaagataagtaactatgatcttgtacaatatgagacacttagtagaaccttaagattaattgttagtgataagat

SEQ ID NO: 107 - DNA, 75 bp, Bovine parainfluenza virus 3
aaaggtaagtaactatgatcttgtacaatatgagacacttagtagaaccttaagattaattgttagtgataagat

SEQ ID NO: 108 - DNA, 75 bp, Bovine parainfluenza virus 3; identical to SEQ ID NO: 107

SEQ ID NO: 109 - DNA, 75 bp, Bovine parainfluenza virus 3
taagtatgaagatatgtgctcagtagacctagccatatcattaagacaaaaaatgtggatgcatttatcaggagg

SEQ ID NO: 110 - DNA, 75 bp, Bovine parainfluenza virus 3
aaagtatgaagacatgtgctcggtagatctagctatctcgttaagacaaaaaatgtggatgcatttatcaggagg

SEQ ID NO: 111 - DNA, 75 bp, Bovine parainfluenza virus 3; identical to SEQ ID NO: 110

SEQ ID NO: 112 - DNA, 75 bp, Bovine parainfluenza virus 3; identical to SEQ ID NO: 110

SEQ ID NO: 113 - DNA, 75 bp, Bovine parainfluenza virus 3
aagaatgataaatggacttgaaactccagatcctttagagttactgtctggagtaataataacaggatctgaaca

SEQ ID NO: 114 - protein, 540 aa, Artificial Sequence
  BPIV3 Fusion Mosaic polypeptide
MIITVATTVILLLPLSLCQIDITKLQRVGVLVNNPKGMKISQNFETRYLILSLIPKIENSHSCGDQQINQYKKLLDRLIIPLYDGLKLQKDVIVVRHESHNNTNSRTKRFFGEIIGTIAIGIATSAQITAAVALVEAKQARSDIDKLKEAIRDTNKAVQSIQSSVGNLIVAVKSVQDYVNNEIVPSITRLGCEAAGLQLGIALTQHYSELTNIFGDNIGTLKEKGIKLQGIASLYRTNITEIFTTSTVDQYDIYDLLFTESIKMRVIDVDLSDYSITLQVRLPLLTKLSNTQIYKVDSISYNIQGKEWYIPLPNHIMTKGAFLGGADIKECIEAFSSYICPSDPGFILNHEIENCLSGNITQCPKTVVTSDIVPRYAFVNGGVIANCIPTTCTCNGIDNRINQSPDQGIKIITHKECQVIGINGMLFRTNKEGTLATYTFDDIVLNNSVALNPIDISMELNKAKLELEESKEWIKKSNQKLDSVGSWYQSSATITIIIVMIIILFIINMIIIIIIIKQFRIRNRHPNDKSNEPYVLTRRQ

SEQ ID NO: 115 - protein, 571 aa, Artificial Sequence
  BPIV3 HN Mosaic polypeptide
MYWRHTNNAKNTNTEFQTETTRRNNKVTNIMMSIFGAILSTILLTVFIMILVGLIQESNHNKIASQQMRREFAEIEKKIQQAADEIGTSIQSGINTRLLTIQSHVQNYIPLSLTQQMSDLRKFINELANKRDQQEVPIQRMTHDSGIEPLNPDKFWRCTSGNPSLTSNPKIRLIPGPSLLAASTTVNGCIRIPSFVINNLIYAYTSNLIVQGCQDIGKSYQVLQIGIITINSDLVPDLNPRVTHTFNIDDNRKSCSLALLNTDVYQLCSTPKVDERSDYASTGIEDIVLDIITNNGLIITTRFTNDNITFDKPYAALYPSVGPGIYYKGKVIFLGYGGLEHAENGDVICNLTGCPGKTQRDCNQASYSPWFSDRRMVNSIIVVNKGVDTTFNLRVWTIPMRQNYWGSEGRLLLLGNKIYIYTRSTSWHSKLQLGTIDITNYSDIRINWTWHDALSRPGNDDCPWGHSCPDGCITGVYTDAYPLNPSGSVVSSVILDSRKSRENPIITYATDTRRVNELAIYNRTLPAAYTTTNCIMHYDKGYCFHIVEINHRSLNTFQPMLFKTEIPKNCS

SEQ ID NO: 116 - protein, 1170 aa, Artificial Sequence
  Chimeric F2 and HN2 polypeptide
MITIVATTVILLLPLSLCQIDTRDYKDDDDKMIITVATTVILLLPLSLCQIDITKLQRVGVLVNNPKGMKISQNFETRYLILSLIPKIENSHSCGDQQINQYKKLLDRLIIPLYDGLKLQKDVIVVRHESHNNTNSRTKRFFGEIIGTIAIGIATSAQITAAVALVEAKQARSDIDKLKEAIRDTNKAVQSIQSSVGNLIVAVKSVQDYVNNEIVPSITRLGCEAAGLQLGIALTQHYSELTNIFGDNIGTLKEKGIKLQGIASLYRTNITEIFTTSTVDQYDIYDLLFTESIKMRVIDVDLSDYSITLQVRLPLLTKLSNTQIYKVDSISYNIQGKEWYIPLPNHIMTKGAFLGGADIKECIEAFSSYICPSDPGFILNHEIENCLSGNITQCPKTVVTSDIVPRYAFVNGGVIANCIPTTCTCNGIDNRINQSPDQGIKIITHKECQVIGINGMLFRTNKEGTLATYTFDDIVLNNSVALNPIDISMELNKAKLELEESKEWIKKSNQKLDSVGSWYQSSATITIIIVMIIILFIINMIIIIIIIKQFRIRNRHPNDKSNEPYVLTRRQGSGATNFSLLKQAGDVEENPGPMYWRHTNNAKNTNTEFQTETTRRNNKVTNIMMSIFGAILSTILLTVFIMILVGLIQESNHNKIASQQMRREFAEIEKKIQQAADEIGTSIQSGINTRLLTIQSHVQNYIPLSLTQQMSDLRKFINELANKRDQQEVPIQRMTHDSGIEPLNPDKFWRCTSGNPSLTSNPKIRLIPGPSLLAASTTVNGCIRIPSFVINNLIYAYTSNLIVQGCQDIGKSYQVLQIGIITINSDLVPDLNPRVTHTFNIDDNRKSCSLALLNTDVYQLCSTPKVDERSDYASTGIEDIVLDIITNNGLIITTRFTNDNITFDKPYAALYPSVGPGIYYKGKVIFLGYGGLEHAENGDVICNLTGCPGKTQRDCNQASYSPWFSDRRMVNSIIVVNKGVDTTFNLRVWTIPMRQNYWGSEGRLLLLGNKIYIYTRSTSWHSKLQLGTIDITNYSDIRINWTWHDALSRPGNDDCPWGHSCPDGCITGVYTDAYPLNPSGSVVSSVILDSRKSRENPIITYATDTRRVNELAIYNRTLPAAYTTTNCIMHYDKGYCFHIVEINHRSLNTFQPMLFKTEIPKNCSHHHHHH

(19) United States
(12) Patent Application Publication
(10) Pub. No.: US 2023/0029979 A1
(43) Pub. Date: Feb. 2, 2023

(54) BROADLY PROTECTIVE BOVINE PARAINFLUENZA 3 VIRUS AND BOVINE VIRAL DIARRHEA VIRUS VACCINE

(71) Applicant: Kansas State University Research Foundation, Manhattan, KS (US)

(72) Inventors: Waithaka Mwangi, Manhattan, KS (US); Huldah Sang, Manhattan, KS (US)

(21) Appl. No.: 17/755,359

(22) PCT Filed: Oct. 30, 2020
(86) PCT No.: PCT/US2020/070725

§ 371 Date: Apr. 27, 2022

Related U.S. Application Data
(60) Provisional application No. 62/928,086, filed on Oct. 30, 2019.

(51) Int. Cl. C12N 15/86

(52) U.S. Cl. CPC C12N 15/86; C12N 2760/18634; C12N 2760/18643

(57) ABSTRACT

A vector comprising a BPI3Vc backbone and at least one antigenic insert sequence from a pathogen other than BPI3V is provided. The vector is configured to provide protection against BPI3V as well as against the pathogen from which the insert sequence was obtained.

### CROSS REFERENCE TO RELATED APPLICATIONS

This application is a National Phase application of PCT Application No. PCT/US2020/070725, filed Oct. 30, 2020, which claims priority to U.S. Provisional Application No. 62/928,086, filed Oct. 30, 2019, the teachings and content of which is incorporated herein in its entirety.

### STATEMENT REGARDING FEDERALLY SPONSORED RESEARCH & DEVELOPMENT

None.

### BACKGROUND OF THE DISCLOSURE

The field of the invention relates generally to immunogenic compositions effective at treating or preventing, or reducing the incidence, severity, frequency, and/or duration of clinical signs of Bovine Respiratory Disease Complex (BRDC) caused by or associated with Bovine Parainfluenza Virus (BPIV) and/or Bovine Viral Diarrhea Virus (BVDV) infection.

Bovine Respiratory Disease Complex (BRDC) leads to severe pneumonia and death of calves and thereby causes multi-billion dollar losses annually due to calf mortality, reduced performance, and use of antibiotics. Several bacterial and viral pathogens are implicated in BRDC. BRDC-associated or causing viral pathogens include Bovine Viral Diarrhea Virus (BVDV) and Bovine Parainfluenza-3 virus (BPI3V). BRDC is characterized by one or more of the following clinical signs or symptoms: fever of over 40° C. (>104° F.), varying degrees of difficulty breathing, nasal discharge, varying degrees of depression (including drooping ears, an extended head, a bowed back, and/or self-isolation from other cattle), diminished or no appetite (‘off-feed’), rapid, shallow breathing, increased lung sounds, coughing, and pneumonia.

Bovine viral diarrhea virus is divided into 2 genotypes, BVDV-1 and BVDV-2, based on significant differences at the level of genomic sequences (summarized in Heinz et al., 2000, hereby entirely incorporated by reference) which are also obvious from limited cross neutralizing antibody reactions (Ridpath et al. 1994, entirely incorporated by reference). BVDV-1 and BVDV-2 cause bovine viral diarrhea (BVD) and mucosal disease (MD) in cattle (Baker, 1987; Moennig and Plagemann, 1992; Thiel et al., 1996, hereby entirely incorporated by reference). Inactivation of the RNase activity residing within the E^{ms} results in an attenuated apathogenic BVDV which may be used as a modified live vaccine (WO 99/64604, hereby entirely incorporated by reference). The international patent application WO2005/111201 (hereby entirely incorporated by reference) provides a further generation of an attenuated BVDV suitable for MLV vaccines, which comprises a multiple modified BVDV having at least one mutation in the coding sequence for glycoprotein E^{ms} and at least another mutation in the coding sequence for N^{pro}, wherein said mutation in the coding sequence for glycoprotein E^{ms} leads to inactivation of RNase activity residing in E^{ms} and/or said mutation in the coding sequence for N^{pro} leads to inactivation of said N^{pro}. Furthermore, various conventional attenuated BVDV viruses are known in the art, which are also suitable candidates for vaccine development. Current BVDV vaccines do not confer broad protection, have immunosuppressive traits, and can induce alloreactive pancytopenia in neonates. Congenital infections may cause resorption, abortion, stillbirth, or live-birth. Congenitally infected fetuses that survive in utero infection (i.e., the live-births) may be born as BVDV-infected calves. The BVDV infection in these calves will persist during the entire life of the calf, and they will shed BVDV continuously in the farm environment. Symptoms of BVDV infection can be variable in adult cattle. Signs of acute infection include fever, lethargy, loss of appetite, ocular discharge, nasal discharge, oral lesions, diarrhea and decreasing milk production. Chronic infection may lead to signs of mucosal disease. In calves, the most commonly recognized birth defect is cerebellar hypoplasia. The signs of this include ataxia/lack of voluntary coordination of muscle movements, tremors, a wide stance, stumbling, failure to nurse, and any combination thereof. In severe cases the calf may die. Transient infections include diarrhea, calf pneumonia, decreased milk production, reproductive disorders, increased occurrence of other diseases, and death. The losses from fetal infection include abortions, congenital defects, weak and abnormally small calves, unthrifty, persistently infected (PI) animals, and death among PI animals.

Bovine Parainfluenza-3 virus (BPI3V) is an RNA virus classified in the paramyxovirus family. BPI3V includes three genotypes, BPI3Va, BPI3Vb, and BPI3Vc. Infections caused by BPI3V are common in cattle. Although BPI3V is capable of causing disease, it is usually associated with mild to subclinical infections. The most important role of BPI3V is to serve as an initiator that may lead to the development of secondary bacterial pneumonia. Clinical signs include pyrexia, cough, serous nasal and lacrimal discharge, increased respiratory rate, and increased breath sounds. The severity of clinical signs worsen with the onset of bacterial pneumonia. Fatalities from uncomplicated BPI3V pneumonia are rare. Lesions include cranioventral lung consolidation, bronchiolitis, and alveolitis with marked congestion and haemorrhage. Inclusion bodies may be identified. Most fatal cases will also have a concurrent bacterial bronchopneumonia. As with BVDV, the current commercial BPI3V vaccines, which are based on a BPI3Va strain, are only effective against homologous strains and closely related variants.

Given that there has been a significant increase in emerging variant BVDV and BPI3V field strains, such as BPIV3c, what is needed are contemporary vaccines and immunogenic compositions effective at improving BRDC management in cattle. Such management would include preventing or treating BPI3V and/or BVDV infections or clinical signs or symptoms thereof. Such prevention and/or treatment would include at least one immunogenic effect selected from the group consisting of reducing the incidence of BPI3V and/or BVDV infection or clinical signs or symptoms thereof in an animal or group of animals, reducing the severity of BPI3V and/or BVDV infection or clinical signs or symptoms thereof in an animal or group of animals, reducing the frequency of BPI3V and/or BVDV infection or clinical signs or symptoms thereof in an animal or group of animals, reducing the duration of BPI3V and/or BVDV infection or clinical signs or symptoms thereof in an animal or group of animals, and any combination thereof.

### BRIEF DESCRIPTION OF THE DISCLOSURE

The present disclosure overcomes the problems encountered in the treatment and/or prevention of BPI3V and/or BVDV infection or clinical signs or symptoms thereof in an animal or group of animals by providing a next generation vaccine or immunogenic composition for induction of broad protection against both BVDV-1 & 2 and BPI3V strains circulating in U.S cattle herds. In general, the vaccine or immunogenic composition comprises, consists essentially of, or consists of a mutant BPI3Vc replicon vector. In some forms, the BPI3Vc replicon is adapted for mucosal or parenteral antigen delivery.

The vaccine or immunogenic composition preferably comprises the mutant BPI3Vc replicon vector as an antigen delivery vector because BPI3Vc strains are now more prevalent in U.S. Advantageously, the mutant BPI3Vc vector can also be used as a platform for other broadly protective vaccines via antigen delivery.

U.S. BPI3Vc isolates include KJ647285.1 Bovine parainfluenza 3 virus isolate TVMDL16, and KJ647287.1 Bovine parainfluenza 3 virus isolate TVMDL20. In China, BPI3Vc isolates include HQ530153.1 and KT071671.1. A BPI3Vc isolate in South Korea is JX969001.1 and a BPI3Vc isolate in Japan is LC000638.1. In some forms, the vector of the present disclosure will have at least 80% homology to one or more of these BPI3Vc isolates. More preferably, the homology will be at least 81, 82, 83, 84, 85, 86, 87, 88, 89, 90, 91, 92, 93, 94, 95, 96, 97, 98, 99% homologous to at least one of these BPI3Vc isolates. More preferably, the vector will be a mosaic of a combination of these BPI3Vc isolates.

In some forms, the BPI3Vc replicon vector includes an insert sequence from a disease-causing organism. In some forms, the organism is BVDV. In some forms, the BVDV insert sequence is at least one subunit of BVDV. In some forms, the subunit is selected from the group consisting of F, HN2, NS2, NS3, NS4, NS5, E2, or any combination thereof.

The BPI3Vc replicon vector of the present disclosure can be administered to an animal in need thereof by any convention method including mucosal or parenteral delivery.

An “immunogenic or immunological composition” refers to a composition of matter that comprises at least one antigen which elicits an immunological response in the host of a cellular and/or antibody-mediated immune response to the composition or vaccine of interest. Usually, an “immunological response” includes but is not limited to one or more of the following effects: the production or activation of antibodies, B cells, helper T cells, suppressor T cells, and/or cytotoxic T cells and/or yd T cells, directed specifically to an antigen or antigens included in the composition or vaccine of interest. Preferably, the host will display either a therapeutic or protective immunological response such that resistance to new infection will be enhanced and/or the clinical severity of the disease reduced. Such protection will be demonstrated by either a reduction in the severity or prevalence of, up to and including a lack of symptoms normally displayed by an infected host, a quicker recovery time and/or a lowered viral titer in the infected host.

“Adjuvants” as used herein, can include aluminum hydroxide and aluminum phosphate, saponins e.g., Quil A, QS-21 (Cambridge Biotech Inc., Cambridge Mass.), GPI-0100 (Galenica Pharmaceuticals, Inc., Birmingham, Ala.), water-in-oil emulsion, oil-in-water emulsion, water-in-oil-in-water emulsion. The emulsion can be based in particular on light liquid paraffin oil (European Pharmacopea type); isoprenoid oil such as squalane or squalene oil resulting from theoligomerization of alkenes, in particular of isobutene or decene; esters of acids or of alcohols containing a linear alkyl group, more particularly plant oils, ethyl oleate, propylene glycol di-(caprylate/caprate), glyceryl tri-(caprylate/caprate) or propylene glycol dioleate; esters of branched fatty acids or alcohols, in particular isostearic acid esters. The oil is used in combination with emulsifiers to form the emulsion. The emulsifiers are preferably nonionic surfactants, in particular esters of sorbitan, of mannide (e.g. anhydromannitol oleate), of glycol, of polyglycerol, of propylene glycol and of oleic, isostearic, ricinoleic or hydroxystearic acid, which are optionally ethoxylated, and polyoxypropylene-polyoxyethylene copolymer blocks, in particular the Pluronic products, especially L121. See Hunter et al., The Theory and Practical Application of Adjuvants (Ed.Stewart-Tull, D. E. S.). JohnWiley and Sons, NY, pp 51-94 (1995) and Todd et al., Vaccine 15:564-570 (1997).

For example, it is possible to use the SPT emulsion described on page 147 of “Vaccine Design, The Subunit and Adjuvant Approach” edited by M. Powell and M. Newman, Plenum Press, 1995, and the emulsion MF59 described on page 183 of this same book.

A further instance of an adjuvant is a compound chosen from the polymers of acrylic or methacrylic acid and the copolymers of maleic anhydride and alkenyl derivative. Advantageous adjuvant compounds are the polymers of acrylic or methacrylic acid which are cross-linked, especially with polyalkenyl ethers of sugars or polyalcohols. These compounds are known by the term carbomer (Phameuropa Vol. 8, No. 2, June 1996). Persons skilled in the art can also refer to U.S. Pat. No. 2,909,462 which describes such acrylic polymers cross-linked with a polyhydroxylated compound having at least 3 hydroxyl groups, preferably not more than 8, the hydrogen atoms of at least three hydroxyls being replaced by unsaturated aliphatic radicals having at least 2 carbon atoms. The preferred radicals are those containing from 2 to 4 carbon atoms, e.g. vinyls, allyls and other ethylenically unsaturated groups. The unsaturated radicals may themselves contain other substituents, such as methyl. The products sold under the name Carbopol; (BF Goodrich, Ohio, USA) are particularly appropriate. They are cross-linked with an allyl sucrose or with allyl pentaerythritol. Among then, there may be mentioned Carbopol 974P, 934P and 971P. Among the copolymers of maleic anhydride and alkenyl derivative, the copolymers EMA (Monsanto) which are copolymers of maleic anhydride and ethylene. The dissolution of these polymers in water leads to an acid solution that will be neutralized, preferably to physiological pH, in order to give the adjuvant solution into which the immunogenic, immunological or vaccine composition itself will be incorporated.

Further suitable adjuvants include, but are not limited to, the RIBI adjuvant system (Ribi Inc.), Block co-polymer (CytRx, Atlanta Ga.), SAF-M (Chiron, Emeryville Calif.), monophosphoryl lipid A, Avridine lipid-amine adjuvant, heat-labile enterotoxin from E. coli (recombinant or otherwise), cholera toxin, IMS 1314 or muramyl dipeptide among many others.

Preferably, the adjuvant is added in an amount of about 100 μg to about 10 mg per dose. Even more preferably, the adjuvant is added in an amount of about 100 μg to about 10 mg per dose. Even more preferably, the adjuvant is added in an amount of about 500 μg to about 5 mg per dose. Even more preferably, the adjuvant is added in an amount of about 750 μg to about 2.5 mg per dose. Most preferably, the adjuvant is added in an amount of about 1 mg per dose.

Additionally, the composition can include one or more pharmaceutical-acceptable or veterinary-acceptable carriers. As used herein, “a pharmaceutical-acceptable carrier” or “veterinary-acceptable carrier” includes any and all solvents, dispersion media, coatings, stabilizing agents, diluents, preservatives, antibacterial and antifungal agents, isotonic agents, adsorption delaying agents, and the like.

The methods of the present disclosure can also comprise the addition of any stabilizing agent, such as for example saccharides, trehalose, mannitol, saccharose and the like, to increase and/or maintain product shelf-life and/or to enhance stability. “Sequence Identity” as it is known in the art refers to a relationship between two or more polypeptide sequences or two or more polynucleotide sequences, namely a reference sequence and a given sequence to be compared with the reference sequence. Sequence identity is determined by comparing the given sequence to the reference sequence after the sequences have been optimally aligned to produce the highest degree of sequence similarity, as determined by the match between strings of such sequences. Upon such alignment, sequence identity is ascertained on a position-by-position basis, e.g., the sequences are “identical” at a particular position if at that position, the nucleotides or amino acid residues are identical. The total number of such position identities is then divided by the total number of nucleotides or residues in the reference sequence to give % sequence identity. Sequence identity can be readily calculated by known methods, including but not limited to, those described in Computational Molecular Biology, Lesk, A. N., ed., Oxford University Press, New York (1988), Biocomputing: Informatics and Genome Projects, Smith, D. W., ed., Academic Press, New York (1993); Computer Analysis of Sequence Data, Part I, Griffin, A. M., and Griffin, H. G., eds., Humana Press, New Jersey (1994); Sequence Analysis in Molecular Biology, von Heinge, G., Academic Press (1987); Sequence Analysis Primer, Gribskov, M. and Devereux, J., eds., M. Stockton Press, New York (1991); and Carillo, H., and Lipman, D., SIAM J. Applied Math., 48: 1073 (1988), the teachings of which are incorporated herein by reference. Preferred methods to determine the sequence identity are designed to give the largest match between the sequences tested. Methods to determine sequence identity are codified in publicly available computer programs which determine sequence identity between given sequences. Examples of such programs include, but are not limited to, the GCG program package (Devereux, J., et al., Nucleic Acids Research, 12(1):387 (1984)), BLASTP, BLASTN and FASTA (Altschul, S. F. et al., J. Molec. Biol., 215:403-410 (1990). The BLASTX program is publicly available from NCBI and other sources (BLAST Manual, Altschul, S. et al., NCVI NLM NIH Bethesda, Md. 20894, Altschul, S. F. et al., J. Molec. Biol., 215:403-410 (1990), the teachings of which are incorporated herein by reference). These programs optimally align sequences using default gap weights in order to produce the highest level of sequence identity between the given and reference sequences. As an illustration, by a polynucleotide having a nucleotide sequence having at least, for example, 85%, preferably 90%, even more preferably 95% “sequence identity” to a reference nucleotide sequence, it is intended that the nucleotide sequence of the given polynucleotide is identical to the reference sequence except that the given polynucleotide sequence may include up to 15, preferably up to 10, even more preferably up to 5 point mutations per each 100 nucleotides of the reference nucleotide sequence. In other words, in a polynucleotide having a nucleotide sequence having at least 85%, preferably 90%, even more preferably 95% identity relative to the reference nucleotide sequence, up to 15%, preferably 10%, even more preferably 5% of the nucleotides in the reference sequence may be deleted or substituted with another nucleotide, or a number of nucleotides up to 15%, preferably 10%, even more preferably 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence. Analogously, by a polypeptide having a given amino acid sequence having at least, for example, 85%, preferably 90%, even more preferably 95% sequence identity to a reference amino acid sequence, it is intended that the given amino acid sequence of the polypeptide is identical to the reference sequence except that the given polypeptide sequence may include up to 15, preferably up to 10, even more preferably up to 5 amino acid alterations per each 100 amino acids of the reference amino acid sequence. In other words, to obtain a given polypeptide sequence having at least 85%, preferably 90%, even more preferably 95% sequence identity with a reference amino acid sequence, up to 15%, preferably up to 10%, even more preferably up to 5% of the amino acid residues in the reference sequence may be deleted or substituted with another amino acid, or a number of amino acids up to 15%, preferably up to 10%, even more preferably up to 5% of the total number of amino acid residues in the reference sequence may be inserted into the reference sequence. These alterations of the reference sequence may occur at the amino or the carboxy terminal positions of the reference amino acid sequence or anywhere between those terminal positions, interspersed either individually among residues in the reference sequence or in the one or more contiguous groups within the reference sequence. Preferably, residue positions which are not identical differ by conservative amino acid substitutions. However, conservative substitutions are not included as a match when determining sequence identity.

“Sequence homology”, as used herein, refers to a method of determining the relatedness of two sequences. To determine sequence homology, two or more sequences are optimally aligned, and gaps are introduced if necessary. However, in contrast to “sequence identity”, conservative amino acid substitutions are counted as a match when determining sequence homology. In other words, to obtain a polypeptide or polynucleotide having 95% sequence homology with a reference sequence, 85%, preferably 90%, even more preferably 95% of the amino acid residues or nucleotides in the reference sequence must match or comprise a conservative substitution with another amino acid or nucleotide, or a number of amino acids or nucleotides up to 15%, preferably up to 10%, even more preferably up to 5% of the total amino acid residues or nucleotides, not including conservative substitutions, in the reference sequence may be inserted into the reference sequence. Preferably the homologous sequence comprises at least a stretch of 50, even more preferably 100, even more preferably 250, even more preferably 500 nucleotides.

A “conservative substitution” refers to the substitution of an amino acid residue or nucleotide with another amino acid residue or nucleotide having similar characteristics or properties including size, hydrophobicity, etc., such that the overall functionality does not change significantly. Those of skill in the art will understand that the composition herein may incorporate known injectable, physiologically acceptable, sterile solutions. For preparing a ready-to-use solution for parenteral injection or infusion, aqueous isotonic solutions, such as e.g. saline or corresponding plasma protein solutions are readily available. In addition, the immunogenic and vaccine compositions of the present disclosure can include diluents, isotonic agents, stabilizers, or adjuvants. Diluents can include water, saline, dextrose, ethanol, glycerol, and the like. Isotonic agents can include sodium chloride, dextrose, mannitol, sorbitol, and lactose, among others. Stabilizers include albumin and alkali salts of ethylendiamintetracetic acid, among others. Suitable adjuvants, are those described above.

According to a further aspect, the immunogenic composition of the present disclosure further comprises a pharmaceutical acceptable salt, preferably a phosphate salt in physiologically acceptable concentrations. Preferably, the pH of said immunogenic composition is adjusted to a physiological pH, meaning between about 6.5 and 7.5.

The immunogenic compositions described herein can further include one or more other immunomodulatory agents such as, e. g., interleukins, interferons, or other cytokines. The immunogenic compositions can also include Gentamicin and Merthiolate. In another preferred embodiment, the present disclosure contemplates vaccine compositions comprising from about 1 ug/ml to about 60 μg/ml of antibiotics, and more preferably less than about 30 μg/ml of antibiotics.

In some forms of the disclosure, a composition for treating, preventing, or reducing the severity, duration, or incidence of clinical signs, is provided. In general, the composition comprises the BPI3Vc replicon vector as described herein and it preferably further includes an insert sequence from a disease-causing organism. In some forms, the organism is BVDV. In some forms, the BVDV insert sequence is from at least one subunit of BVDV. In some forms, the subunit is selected from the group consisting of F, HN2, NS2, NS3, NS4, NS5, E2, or any combination thereof. In some forms, the subunit is selected from nucleotide sequences encoding sequences having at least 80, 85, 86, 87, 88, 89, 90, 91, 92, 93, 94, 95, 96, 97, 98, 99, 99.5, or even 100% sequence homology or sequence identity with a sequence selected from the group consisting of SEQ ID NOS. 2, 3, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 72, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, or any combination thereof. In some forms the insert sequence encodes for a mosaic sequence selected from sequences having at least 80, 85, 86, 87, 88, 89, 90, 91, 92, 93, 94, 95, 96, 97, 98, 99, 99.5, or even 100% sequence identity or sequence homology with a sequence selected from the group consisting of SEQ ID NOS. 2, 3, 114, 115, 116, or any combination thereof.

In some forms, the composition is administered with an adjuvant, as described above.

In some forms, the composition is administered with a veterinary-acceptable carrier, as described above.

In some forms, the composition is administered with a stabilizer, as described above.

In some forms, the composition is administered with a pharmaceutical acceptable salt, as described above.

In some forms, the composition is administered with at least one immunomodulatory agent, as described above.

These compositions can be administered by the systemic route, in particular by the intravenous route, by the intramuscular, intradermal or subcutaneous route, or by the oral route. In a more preferred manner, the vaccine composition comprising polypeptides according to the disclosure will be administered by the intramuscular route, through the food or by nebulization several times, staggered over time.

Their administration modes, dosages and optimum pharmaceutical forms can be determined according to the criteria generally taken into account in the establishment of a treatment adapted to an animal such as, for example, the age or the weight, the seriousness of its general condition, the tolerance to the treatment and the secondary effects noted. Preferably, the vaccine of the present disclosure is administered in an amount that is protective or provides a protective effect against BPIV3 infection and preferably, at least one other infection other than BPIV3, such as BVDV.

### BRIEF DESCRIPTION OF THE DRAWINGS

FIG. 1 is a representation of the nucleic acid sequence (SEQ ID NO. 1) of a vector in accordance with the disclosure;

FIG. 2 is a schematic of the vector of the disclosure;

FIG. 3A is a representation of the novel mosaic fusion [F] polypeptide (SEQ ID NO. 2) generated from 62 currently sequenced BPI3V genotypes A, B, and C (polypeptide sequences are from Amino to carboxyl terminus);

FIG. 3B is a representation of the novel mosaic hemagglutinin-neuraminidase [HN] polypeptide (SEQ ID NO. 3) generated from 62 currently sequenced BPI3V genotypes A, B, and C (polypeptide sequences are from Amino to carboxyl terminus);

FIG. 4 is a schematic of an optimized T7 promoter;

FIG. 5 is a photograph of an attenuated BPI3Vc-E2^{b} virus expressing the E2^{b} transgene;

FIG. 6A is a photograph of a BVDV E2^{b} transgene by BPI3Vc-E2^{b} virus on a cell surface using anti-FLAG monoclonal antibody;

FIG. 6B is a photograph of a BVDV E2^{b} transgene by BPI3Vc-E2^{b} virus on a cell surface using BPI3V polyclonal antibody (detects expression of BPI3Vc antigens);

FIG. 6C is a photograph of a BVDV E2^{b} transgene by BPI3Vc-E2^{b} virus on a cell surface using BVDV Type 1&2 monoclonal antibody (mAb 348) against E2;

FIG. 6D is a photograph of a BVDV E2^{b} transgene by BPI3Vc-E2^{b} virus on a cell surface of an uninfected negative control;

FIG. 7A is a photograph illustrating the expression and authenticity of FLAG-tagged mosaic novel fusion [F2] and HIS-tagged Hemagglutinin-Neuraminidase [HN2] proteins evaluated by immunocytometric analysis of HEK-293A cells transfected with plasmid constructs and probed with Anti-FLAG monoclonal antibody to detect the FLAG-tagged Fusion protein;

FIG. 7B is a photograph illustrating the expression and authenticity of FLAG-tagged mosaic novel fusion [F2] and HIS-tagged Hemagglutinin-Neuraminidase [HN2] proteins evaluated by immunocytometric analysis of HEK-293A cells transfected with plasmid constructs and probed with anti-HIS monoclonal antibody to detect the HIS-tagged Hemagglutinin-Neuraminidase protein;

FIG. 7C is a photograph illustrating the expression and authenticity of FLAG-tagged mosaic novel fusion [F2] and HIS-tagged Hemagglutinin-Neuraminidase [HN2] proteins evaluated by immunocytometric analysis of HEK-293A cells transfected with plasmid constructs and probed with) BPI3V polyclonal antibody; and

FIG. 7D is a photograph illustrating the expression and authenticity of FLAG-tagged mosaic novel fusion [F2] and HIS-tagged Hemagglutinin-Neuraminidase [HN2] proteins evaluated by immunocytometric analysis of HEK-293A cells transfected with plasmid constructs and probed with pCDNA construct negative control.

FIG. 8A is a photograph illustrating protein expression by BPI3Vc-E2-NS2-3b virus. Rescued recombinant BPI3Vc-E2-NS2-3b virus was used to infect MDBK cells and at 72 hours post-infection, immunocytometric analysis was used to validate expression of the transgene using Anti-FLAG mAb;

FIG. 8B is a photograph illustrating protein expression by BPI3Vc-E2-NS2-3b virus. Rescued recombinant BPI3Vc-E2-NS2-3b virus was used to infect MDBK cells and at 72 hours post-infection, immunocytometric analysis was used to validate expression of the transgene using BPI3V reference serum;

FIG. 8C is a photograph illustrating protein expression by BPI3Vc-E2-NS2-3b virus. Rescued recombinant BPI3Vc-E2-NS2-3b virus was used to infect MDBK cells and at 72 hours post-infection, immunocytometric analysis was used to validate expression of the transgene using BVDV E2-specific mAb 348;

FIG. 8D is a photograph illustrating protein expression by BPI3Vc-E2-NS2-3b virus. Rescued recombinant BPI3Vc-E2-NS2-3b virus was used to infect MDBK cells and at 72 hours post-infection, immunocytometric analysis was used to validate expression of the transgene using uninfected negative control cells probed with the anti-BPI3V reference serum;

FIG. 9A is a photograph illustrating the validation of expression of rescued recombinant BPI3Vc-NS4-5¹ virus. The virus was used to infect MDBK cells and at 72 hours post infection, immunocytometric analysis was used to validate expression of the transgene using Anti-FLAG mAb;

FIG. 9B is a photograph illustrating the validation of expression of rescued recombinant BPI3Vc-NS4-5¹ virus. The virus was used to infect MDBK cells and at 72 hours post infection, immunocytometric analysis was used to validate expression of the transgene using BPI3V polyclonal reference antibody;

FIG. 9C is a photograph illustrating the validation of expression of rescued recombinant BPI3Vc-NS4-5¹ virus. The virus was used to infect MDBK cells and at 72 hours post infection, immunocytometric analysis was used to validate expression of the transgene using Anti-BVDV polyclonal antibody;

FIG. 9D is a photograph illustrating the validation of expression of rescued recombinant BPI3Vc-NS4-5¹ virus. The virus was used to infect MDBK cells and at 72 hours post infection, immunocytometric analysis was used to validate expression of the transgene using uninfected negative control cells probed with the anti-BPI3V reference serum;

FIG. 10A is a photograph illustrating protein expression by BPI3Vc-F2-HN2 virus. Rescued recombinant BPI3Vc-F2-HN2 virus was used to infect MDBK cells and at 72 hours post-infection, immunocytometric analysis was used to validate expression of the transgenes FLAG-tagged F2 protein using anti-FLAG mAb [probed unfixed cells to detect surface expression];

FIG. 10B is a photograph illustrating protein expression by BPI3Vc-F2-HN2 virus. Rescued recombinant BPI3Vc-F2-HN2 virus was used to infect MDBK cells and at 72 hours post-infection, immunocytometric analysis was used to validate expression of the transgenes His-tagged HN2 using anti-His mAb;

FIG. 10C is a photograph illustrating protein expression by BPI3Vc-F2-HN2 virus. Rescued recombinant BPI3Vc-F2-HN2 virus was used to infect MDBK cells and at 72 hours post-infection, immunocytometric analysis was used to validate expression of the transgenes authenticity of the BPI3Vc, F2, and HN2 proteins using the anti-BPI3V reference serum;

FIG. 10D is a photograph illustrating protein expression by BPI3Vc-F2-HN2 virus. Rescued recombinant BPI3Vc-F2-HN2 virus was used to infect MDBK cells and at 72 hours post-infection, immunocytometric analysis was used to validate expression of the transgenes of uninfected negative control cells probed with the anti-BPI3V reference serum; and

FIG. 11 is a graph illustrating that BPI3Vc-F2HN2 virus is temperature sensitive.

### DETAILED DESCRIPTION OF THE DISCLOSURE

This written description uses examples to describe the disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

### Example 1

This example generates a BPI3Vc backbone for use as a vector and for delivery and/or expression of antigens in an animal in need thereof.

The BPI3V Genotype C strain TVMDL16 was used as a vaccine strain and vector expressing BVDV E2 antigen.

Fully sequenced complete BPI3V genomes in the US were retrieved from NCBI and aligned. They cluster into 3 main clades representing Genotype A, B, and C, which is consistent with previous reports. The NC 002161.1 BPI3V complete genome, AF 178654.1 BPI3V strain Kansas/15626/84 complete genome, AF 178655.1 BPI3V Shipping Fever complete genome, KJ647288.1 BPI3V isolate TVMDL24 complete genome, and KJ647289.1 BPI3V isolate TVMDL60 complete genome were identified as belong to genotype A. The KJ647284.1 BPI3V isolate TVMDL15 complete genome, KP764763.1 BPI3V strain TtPIV-1 complete genome, and KJ647286.1 BPI3V isolate TVMDL17 complete genome were identified as belonging to genotype B. The KJ647285.1 BPI3V isolate TVMDL16 complete genome and KJ647287.1 BPI3V isolate TVMDL20 complete genome were identified as belonging to genotype C.

The BPI3V Genotypes C TVMDL16 and TVMDL20, protein and nucleotide sequences for the following BPI3V Genotype C isolates in different parts of the world were aligned as shown in Table 1.

TABLE 1

| Virus isolate | Accession # | Country |
|---|---|---|
| Bovine parainfluenza virus 3 isolate TVMDL16, complete genome | KJ647285.1 | USA |
| Bovine parainfluenza virus 3 isolate TVMDL20, complete genome | KJ647287.1 | USA |
| Bovine parainfluenza virus 3 strain SD0835, complete genome | HQ530153.1 | China |
| Bovine parainfluenza virus 3 isolate 12Q061, complete genome | JX969001.1 | S. Korea |
| Bovine parainfluenza virus 3 strain NX49, complete genome | KT071671.1 | China |
| Bovine parainfluenza virus 3 viral cRNA, complete genome, isolate: HS9 | LC000638.1 | Japan |

Following alignment, regions where an amino acid from the TVMDL16 strain differed from the TVMDL20 strain were identified. This particular site was compared to the other four aligned sequences to determine the most dominant consensus as exemplified below in Table 2 for the phosphoprotein.

Amino Acid Alignment

TABLE 2
with SEQ ID NOS. 10-33, respectively.

| | |
|---|---|
| AHZ90090.1 phosphoprotein Bovine respirovirus 3 | 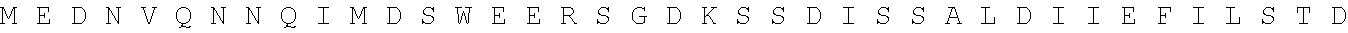 |
| AHZ90102.1 phosphoprotein Bovine respirovirus 3 | M E D N A Q N N Q I M D S W E E R S G D K S S D I S S A L D I I E F I L S T D |
| ADQ43752.1 phosphoprotein P Bovine respirovirus 3 | 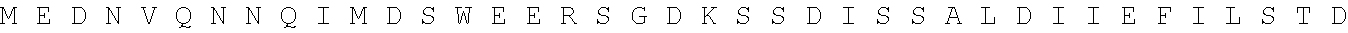 |
| AGA95117.1 phosphoprotein Bovine respirovirus 3 | 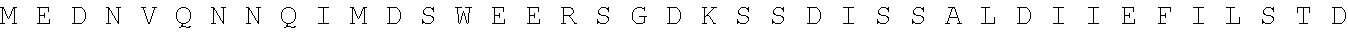 |
| ALS45554.1 phosphoprotein Bovine respirovirus 3 | 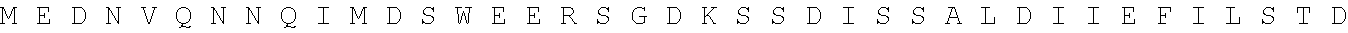 |
| BAP75929.1 phosphoprotein P Bovine respirovirus 3 | 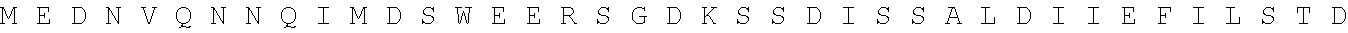 |
| AHZ90090.1 phosphoprotein Bovine respirovirus 3 | 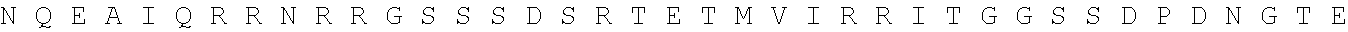 |
| AHZ90102.1 phosphoprotein Bovine respirovirus 3 | N Q E A I Q G R N G R G S S S D S R T E T L V I R R I T G G S S D P D N G T E |
| ADQ43752.1 phosphoprotein P Bovine respirovirus 3 | 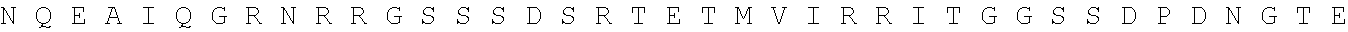 |
| AGA96117.1 phosphoprotein Bovine respirovirus 3 | 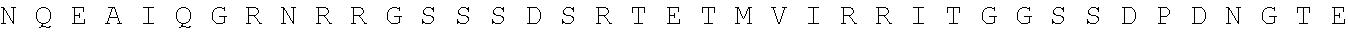 |
| ALS46554.1 phosphoprotein Bovine respirovirus 3 | 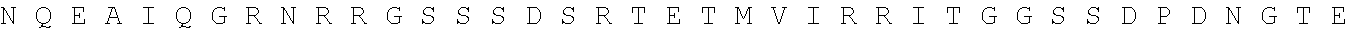 |
| BAP75929.1 phosphoprotein P Bovine respirovirus 3 | 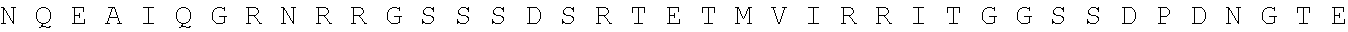 |
| AHZ90090.1 phosphoprotein Bovine respirovirus 3 | 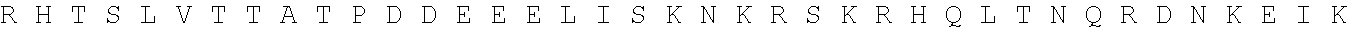 |
| AHZ90102.1 phosphoprotein Bovine respirovirus 3 | R H T S L V T T A T P D D E E E L I L K N K R S K R H Q L T N Q R D N K E I K |
| ADQ43752.1 phosphoprotein P Bovine respirovirus 3 | 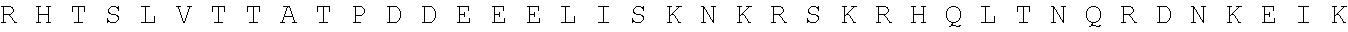 |
| AGA95117.1 phosphoprotein Bovine respirovirus 3 | 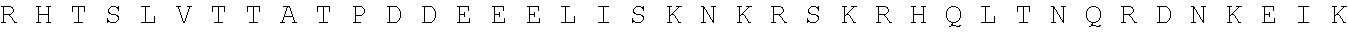 |
| ALS45554.1 phosphoprotein Bovine respirovirus 3 | 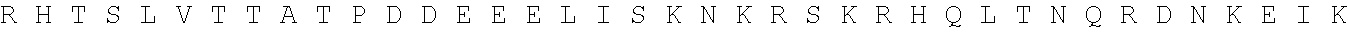 |
| BAP75929.1 phosphoprotein P Bovine respirovirus 3 | 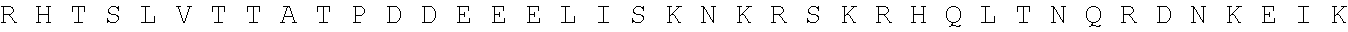 |
| AHZ90090.1 phosphoprotein Bovine respirovirus 3 | 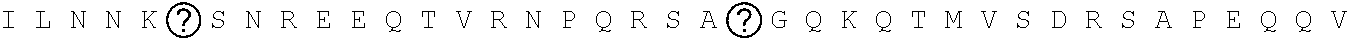 |
| AHZ90102.1 phosphoprotein Bovine respirovirus 3 | I L N N K N S N R E E Q T V R N P Q R S A Y G Q K Q T M V S D R S A P E Q P V |
| ADQ43752.1 phosphoprotein P Bovine respirovirus 3 | 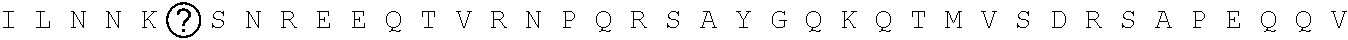 |
| AGA96117.1 phosphoprotein Bovine respirovirus 3 | 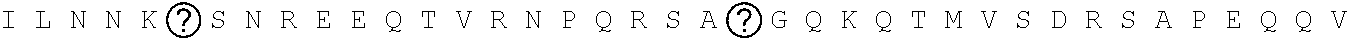 |
| ALS46554.1 phosphoprotein Bovine respirovirus 3 | 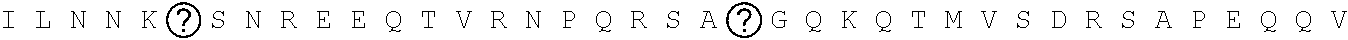 |
| BAP75929.1 phosphoprotein P Bovine respirovirus 3 | 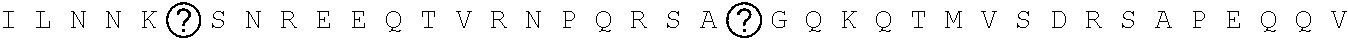 |

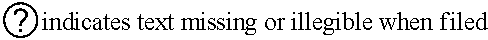

Nucleotide alignment is shown below in Table 3 which includes SEQ ID NOS. 34-57, respectively.

TABLE 3

| | |
|---|---|
| KJ847285.1 Bovine parainfluenza virus 3 isolate TVMDL18 complete genome | 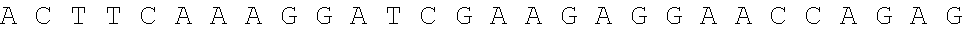 |
| KJ847287.1 Bovine parainfluenza virus 3 isolate TVMDL20 complete genome | A C T T C A A A G G A T C G A A G A G G A G C C A G A G |
| HQ530153.1 Bovine parainfluenza virus 3 strain SD0835 complete genome | 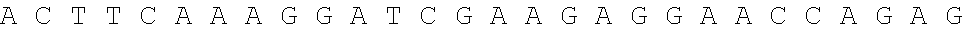 |
| JX969001.1 Bovine parainfluenza virus 3 isolate 12Q081 complete genome | 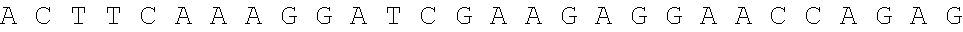 |
| KJ071 [symbol] 71.1 Bovine parainfluenza virus 3 strain N [symbol] 49 complete genome | 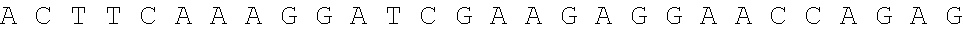 |
| LC000838.1 Bovine parainfluenza virus 3 viral cRNA complete genome isolate H [symbol] | 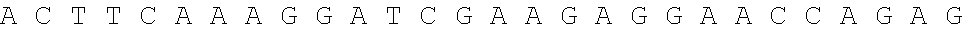 |
| KJ847285.1 Bovine parainfluenza virus 3 isolate TVMDL18 complete genome | 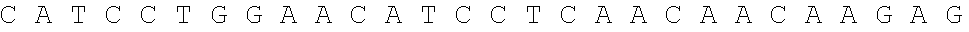 |
| KJ847287.1 Bovine parainfluenza virus 3 isolate TVMDL20 complete genome | C A T C C T G G A A C A T C C T C A A C A A C A A G A A |
| HQ530153.1 Bovine parainfluenza virus 3 strain SD0835 complete genome | 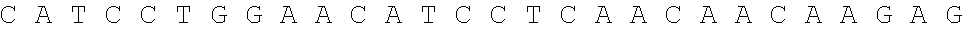 |
| JX969001.1 Bovine parainfluenza virus 3 isolate 12Q081 complete genome | 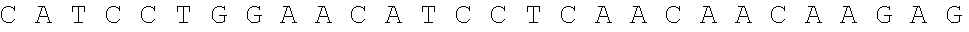 |
| KJ071 [symbol] 71.1 Bovine parainfluenza virus 3 strain N [symbol] 49 complete genome | 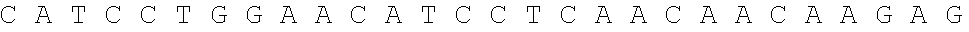 |
| LC000838.1 Bovine parainfluenza virus 3 viral cRNA complete genome isolate H [symbol] | 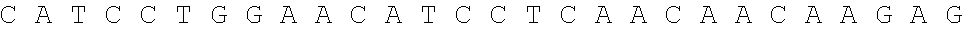 |
| KJ847285.1 Bovine parainfluenza virus 3 isolate TVMDL18 complete genome | 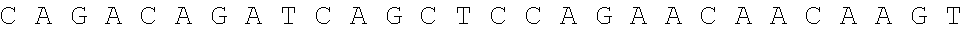 |
| KJ847287.1 Bovine parainfluenza virus 3 isolate TVMDL20 complete genome | C A G A C A G A T C A G C T C C C G A A C A A C C A G T |
| HQ530153.1 Bovine parainfluenza virus 3 strain SD0835 complete genome | 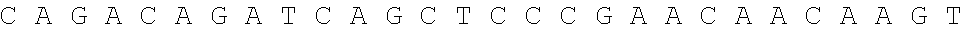 |
| JX969001.1 Bovine parainfluenza virus 3 isolate 12Q081 complete genome | 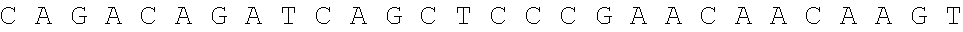 |
| KJ071 [symbol] 71.1 Bovine parainfluenza virus 3 strain N [symbol] 49 complete genome | 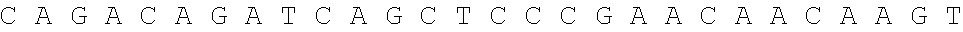 |
| LC000838.1 Bovine parainfluenza virus 3 viral cRNA complete genome isolate H [symbol] | 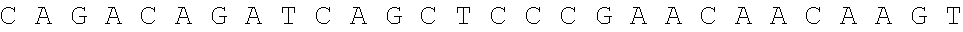 |
| KJ847285.1 Bovine parainfluenza virus 3 isolate TVMDL18 complete genome | 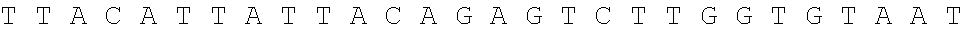 |
| KJ847287.1 Bovine parainfluenza virus 3 isolate TVMDL20 complete genome | T T A C A T T A T T A C A G A A T C T T G G T G T A A T |
| HQ530153.1 Bovine parainfluenza virus 3 strain SD0835 complete genome | 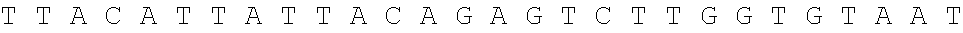 |
| JX969001.1 Bovine parainfluenza virus 3 isolate 12Q081 complete genome | 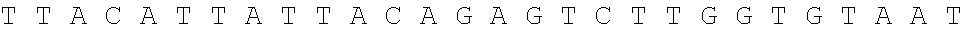 |
| KJ071 [symbol] 71.1 Bovine parainfluenza virus 3 strain N [symbol] 49 complete genome | 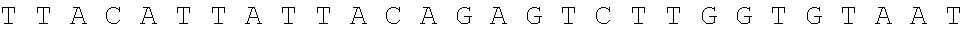 |
| LC000838.1 Bovine parainfluenza virus 3 viral cRNA complete genome isolate H [symbol] | 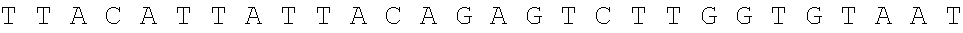 |

[symbol]

indicates data missing or illegible when filed

Based on these alignments, the total number of TVMDL16 or TVMDL20 variable sites that were similar to the rest aligned sequences were added and results obtained as shown in Table 4 below:

TABLE 4

| Protein/Nucleotide | TVMDL16 | TVMDL20 |
|---|---|---|
| Nucleoprotein | 0 | 2 |
| Phosphoprotein | 12 | 2 |
| Matrix | 0 | 0 |
| Fusion | 0 | 1 |
| Hemagglutinin-neuraminidase | 3 | 1 |
| Large polymerase | 7 | 5 |
| Total | 22 | 11 |
| Nucleotide genome Total | 104 | 73 |

Attenuating BPIV-3 TVMDL16 (Mutation “a”).

Attenuation based on mutations obtained from current vaccine strains: The selected BPI3Vc TVMDL16 genome was aligned together with BPI3V vaccine strains in use in the US, Shipping Fever strain and Kansas/15626/84, which are both Genotype A. Other published Genotypes A and C sequences were also included in order to identify specific sites which are conserved only for the US vaccine strains. Table 5 shows the genomes used in this alignment and Table 6 provides the alignment of SEQ ID NOS. 58-85, respectively.

TABLE 5

| Virus isolate | Accession # | Country | Genotype |
|---|---|---|---|
| Bovine parainfluenza virus 3 strain NM09 from China, complete genome | JQ063064.1 | China | A |
| Bovine parainfluenza virus 3 DNA, complete genome | D84095.1 | Japan | A |
| Bovine parainfluenza virus 3 strain Shipping Fever, complete genome | AF178655.1 | US | A |
| Bovine parainfluenza virus 3 isolate TVMDL24, complete genome | KJ647288.1 | US | A |
| Bovine parainfluenza virus 3 isolate TVMDL60, complete genome | KJ647289.1 | US | A |
| Bovine parainfluenza virus 3 viral cRNA, complete genome, strain: BN-1 | AB770484.1 | Japan | A |
| Bovine parainfluenza virus 3 viral cRNA, complete genome, strain: BN-CE | AB770485.1 | Japan | A |
| Bovine parainfluenza virus 3 strain Kansas/15626/84, complete genome | AF178654.1 | US | A |
| Bovine parainfluenza virus 3 isolate TVMDL16, complete genome | KJ647285.1 | USA | C |
| Bovine parainfluenza virus 3 isolate TVMDL20, complete genome | KJ647287.1 | USA | C |
| Bovine parainfluenza virus 3 strain SD0835, complete genome | HQ530153.1 | China | C |
| Bovine parainfluenza virus 3 isolate 12Q061, complete genome | JX969001.1 | S. Korea | C |
| Bovine parainfluenza virus 3 strain NX49, complete genome | KT071671.1 | China | C |
| Bovine parainfluenza virus 3 viral cRNA, complete genome, isolate: HS9 | LC000638.1 | Japan | C |

Specific amino acids variable only for the vaccine strains in US but conserved across the Genotypes A and C were identified below in Table 6.

TABLE 6

| | |
|---|---|
| AEU04137.1 phosphoprotein Bovine respirovirus 3 | 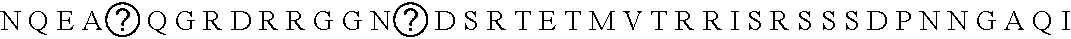 |
| BAA12214.1 P protein Bovine respirovirus 3 | 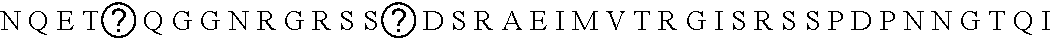 |
| AAF28261.1 phosphoprotein P Bovine respirovirus 3 | 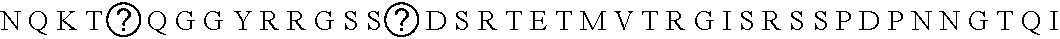 |
| AHZ90108.1 phosphoprotein Bovine respirovirus 3 | 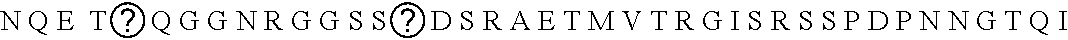 |
| AHZ90114.1 phosphoprotein Bovine respirovirus 3 | 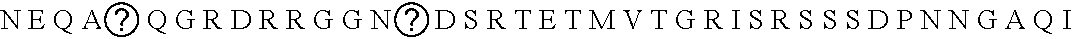 |
| BAM72618.1 phosphoprotein P Bovine respirovirus 3 | 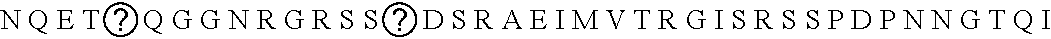 |
| BAM72624.1 phosphoprotein P Bovine respirovirus 3 | 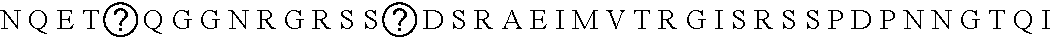 |
| AAF28255.1 phosphoprotein P Bovine respirovirus 3 | 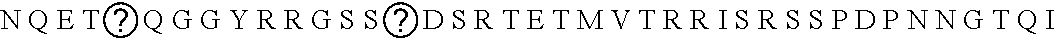 |
| AHZ90090.1 phosphoprotein Bovine respirovirus 3 | 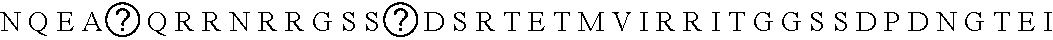 |
| AHZ90102.1 phosphoprotein Bovine respirovirus 3 | 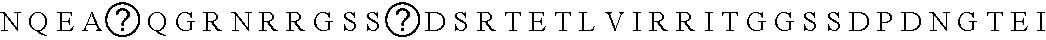 |
| ADQ43752.1 phosphoprotein P Bovine respirovirus 3 | 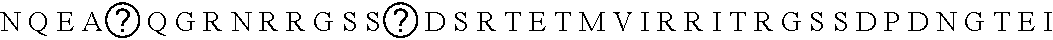 |
| AGA96117.1 phosphoprotein Bovine respirovirus 3 | 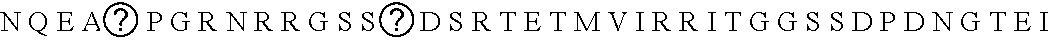 |
| ALS46554.1 phosphoprotein Bovine respirovirus 3 | 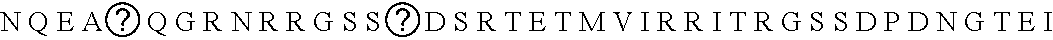 |
| BAP75929.1 phosphoprotein P Bovine respirovirus 3 | 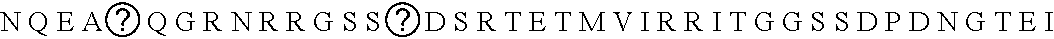 |
| | 105 115 |
| AEU04137.1 phosphoprotein Bovine respirovirus 3 | 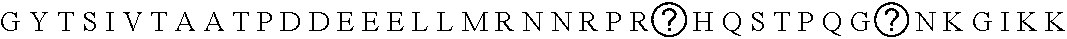 |
| BAA12214.1 P protein Bovine respirovirus 3 | 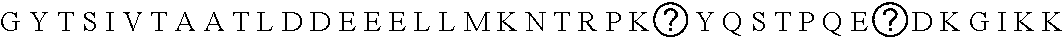 |
| AAF28261.1 phosphoprotein P Bovine respirovirus 3 | 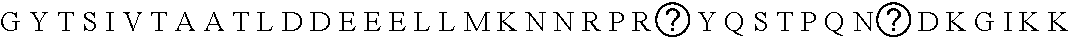 |
| AHZ90108.1 phosphoprotein Bovine respirovirus 3 | 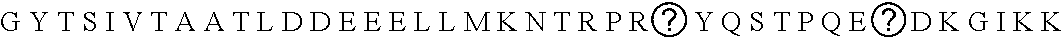 |
| AHZ90114.1 phosphoprotein Bovine respirovirus 3 | 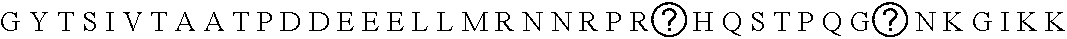 |
| BAM72618.1 phosphoprotein P Bovine respirovirus 3 | 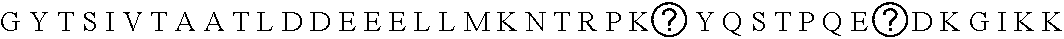 |
| BAM72624.1 phosphoprotein P Bovine respirovirus 3 | 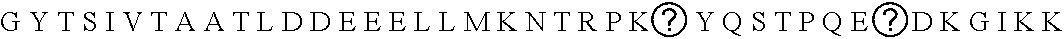 |
| AAF28255.1 phosphoprotein P Bovine respirovirus 3 | 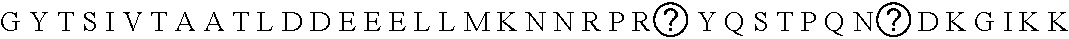 |
| AHZ90080.1 phosphoprotein Bovine respirovirus 3 | 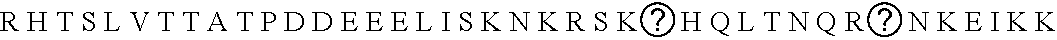 |
| AHZ90102.1 phosphoprotein Bovine respirovirus 3 | 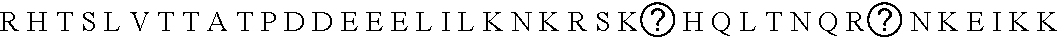 |
| ADQ43752.1 phosphoprotein P Bovine respirovirus 3 | 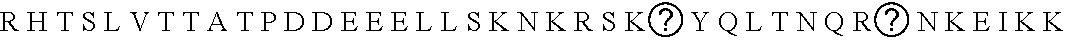 |
| AGA96117.1 phosphoprotein Bovine respirovirus 3 | 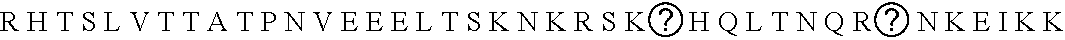 |
| ALS46554.1 phosphoprotein Bovine respirovirus 3 | 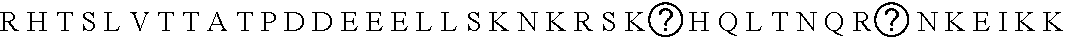 |
| BAP75929.1 phosphoprotein P Bovine respirovirus 3 | 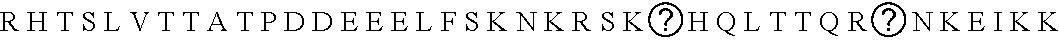 |

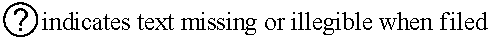

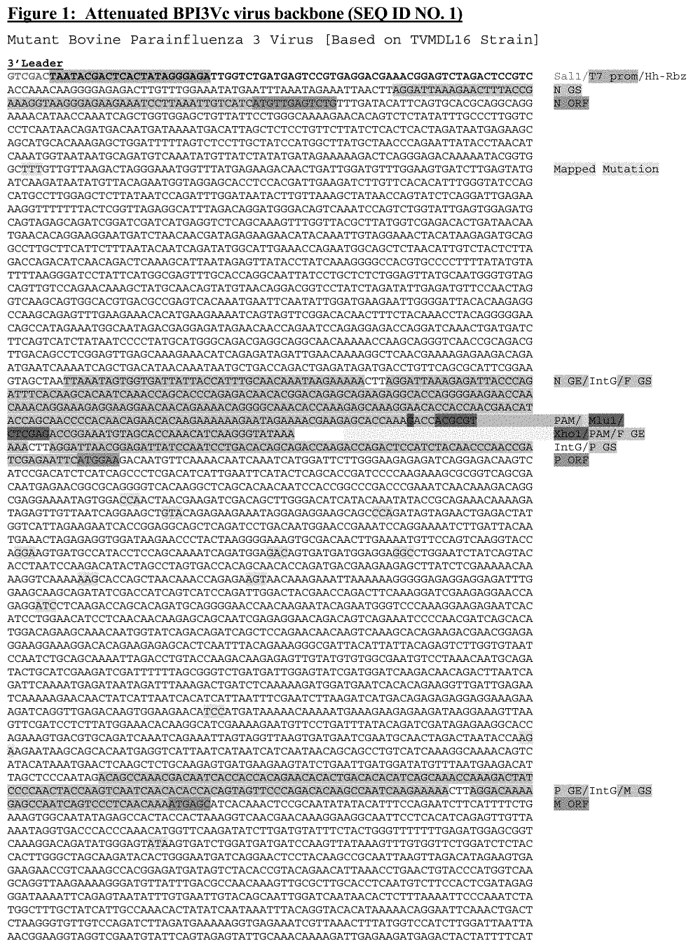

A sample of these sites that formed the basis of creating exact mutations on the TVMDL16 strain to create a mutant BPI3V TVMDL16 is shown below in Table 7, which includes SEQ ID NOS. 86-100, respectively. The complete mutated sequence is provided in the sequence listing as SEQ ID NO. 1 and is shown in FIG. 1.

TABLE 7

| | |
|---|---|
| [symbol] | C A A A A C A A C A G A A A C A A G C A A G G A A A A T A G T G G A C C A G C T A A C A A A A A T C G A C A G T T T G |
| [symbol] | 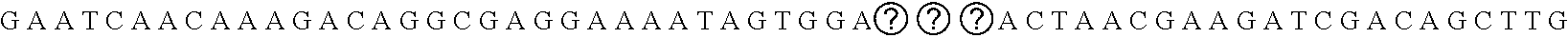 |
| [symbol] | G A A A T C A A C A A A G A C A G G C G A G G A A A A T A G T G G A C C A A C T A A C G A A G A T C G A C A G C T T G |
| [symbol] | A G A T A G A A A T G T T A A T C A G G A G A C T G T A C A G G G A G A A T A T A G G A G A G G A A G C A G C C C A G |
| [symbol] | 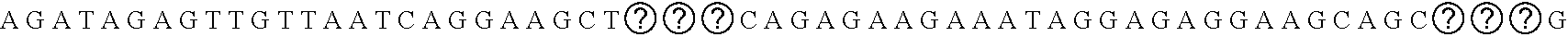 |
| [symbol] | A G A T A G A G T T G T T A A T C A G G A A G C T G T A C A G A G A A G A A A T A G G A G A G G A A G C A G C C C A G |
| [symbol] | A A T C T C C A G A A G C A G C C C A G A T C C T A A C A A T G G A A C C C A A A T C C A G G A A G A T A T T G A T T |
| [symbol] | A A T C A C C G G A G G C A G C T C A G A T C C T G A C A A T G G A A C C G A A A T C C A G G A A A A T C T T G A T T |
| [symbol] | A A T C A C C G G A G G C A G C T C A G A T C C T G A C A A T G G A A C C G A A A T C C A G G A A A A T C T T G A T T |
| [symbol] | 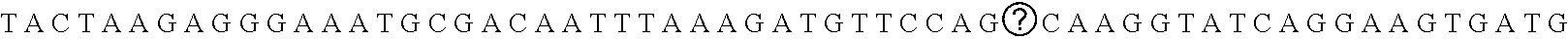 |
| [symbol] | 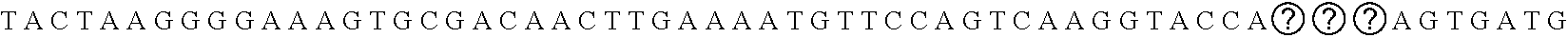 |
| [symbol] | T A C T A A G G G G A A A G T G C G A C A A C T T G A A A A T G T T C C A G T C A A G G T A C C A G G A A G T G A T G |
| [symbol] | T G A T G A T G G A A G A G G C C T G G A A T C T A T C A G T A C A T T T G A T T C A G G A T A T A C C A G T A T A G |
| [symbol] | T G A T G A T G G A G G A A G C C T G G A A T C T A T C A G T A C A C C T A A T C C A A G A C A T A C T A G C C T A G |
| [symbol] | T G A T G A T G G A G G A G G C C T G G A A T C T A T C A G T A C A C C T A A T C C A A G A C A T A C T A G C C T A G |

[symbol]

indicates data missing or illegible when filed

Temperature sensitive attenuating mutation (mutation “b”). A distinct temperature sensitive single substitution in the polymerase gene, I 1103 V (change from Isoleucine to Valine in position 1103) was previously identified to cause temperature sensitive (ts) and attenuated phenotype in the reference Kansas/15626/84 vaccine strain. In this regard, this substitution was also made in some forms of the mutant BPI3V TVMDL16 genome at position 1103 from Isoleucine (ATA) to Valine (GTA).

The combination of mutation a and mutation b form a preferred form of the BPI3Vc vector platform shown below in Table 8, which includes SEQ ID NOS. 101-113, respectively.

TABLE 8

| | |
|---|---|
| [symbol] | T G G T A T G T T G G A T A C A A C A A A A T C A C T A A T T C G A G T A G G G A T A A G C A G A G G A G G A T T A A C C T A T A A C T T A T T A A G |
| [symbol] | A G G A A T G C T G G A C A C A A C A A A A T C G T T A A T T C G A G T A G G G A T A A A T C G A G G A G G G T T A A C T T A T A G T T T G T T A A G |
| [symbol] | A G G A A T G C T G G A C A C A A C A A A A T C G T T A A T T C G A G T A G G G A T A A A T C G A G G A G G G T T A A C T T A T A G T T T G T T A A G |
| [symbol] | A G G A A T G C T G G A C A C A A C A A A A T C G T T A A T T C G A G T A G G G A T A A A T C G A G G A G G G T T A A C T T A T A G T T T G T T A A G |
| [symbol] | 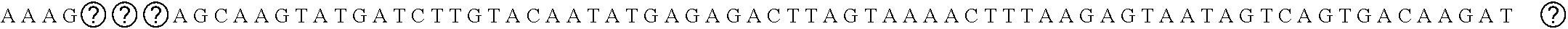 |
| [symbol] | 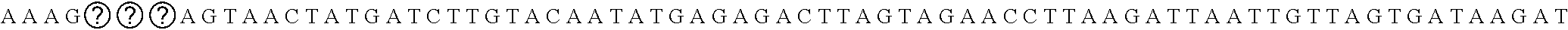 |
| [symbol] | 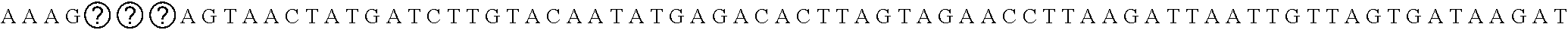 |
| [symbol] | 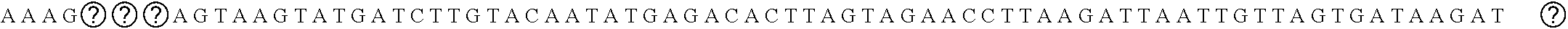 |
| [symbol] | T A A G T A T G A A G A T A T G T G C T C A G T A G A C C T A G C C A T A T G A T T A A G A C A A A A A A T G T G G A T G G A T T T A T C A G G A G G |
| [symbol] | A A A G T A T G A A G A C A T G T G C T C G G T A G A T C T A G C T A T C T C G T T A A G A C A A A A A A T G T G G A T G C A T T T A T C A G G A G G |
| [symbol] | A A A G T A T G A A G A C A T G T G C T C G G T A G A T C T A G C T A T C T C G T T A A G A C A A A A A A T G T G G A T G C A T T T A T C A G G A G G |
| [symbol] | A A A G T A T G A A G A C A T G T G C T G G G T A G A T C T A G C T A T C T C G T T A A G A C A A A A A A T G T G G A T G C A T T T A T C A G G A G G |
| [symbol] | A A G A A T G A T A A A T G G A C T T G A A A C T C G A G A T C C T T T A G A G T T A C T G T C T G G A G T A A T A A T A A C A G G A T C T G A A C A |

[symbol]

indicates data missing or illegible when filed

The above mentioned modifications (a and b) created the ‘Mutant BPI3V TVMDL16 genome.

Design of a Vaccine Vector from Mutant BPIV3 TVMDL16 Genome.

Insert position and design: BPI3Va has previously been used as a vaccine vector for expressing foreign proteins of Human parainfluenza virus-3 and Respiratory syncytial virus, while being able to retain its infectivity and immunogenicity. The position of insertion in the parainfluenza virus genome determines the level of expression of gene of interest. Higher levels of expression are observed with inserts placed at closer to the 3′ end of the negative sense genome and level of expression decreases with downstream insert positions.

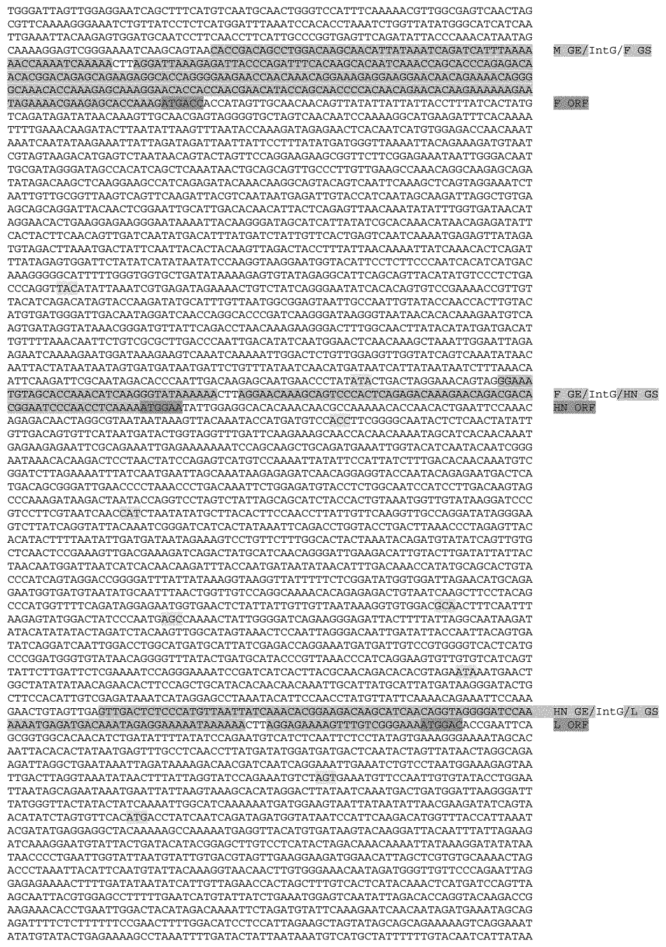

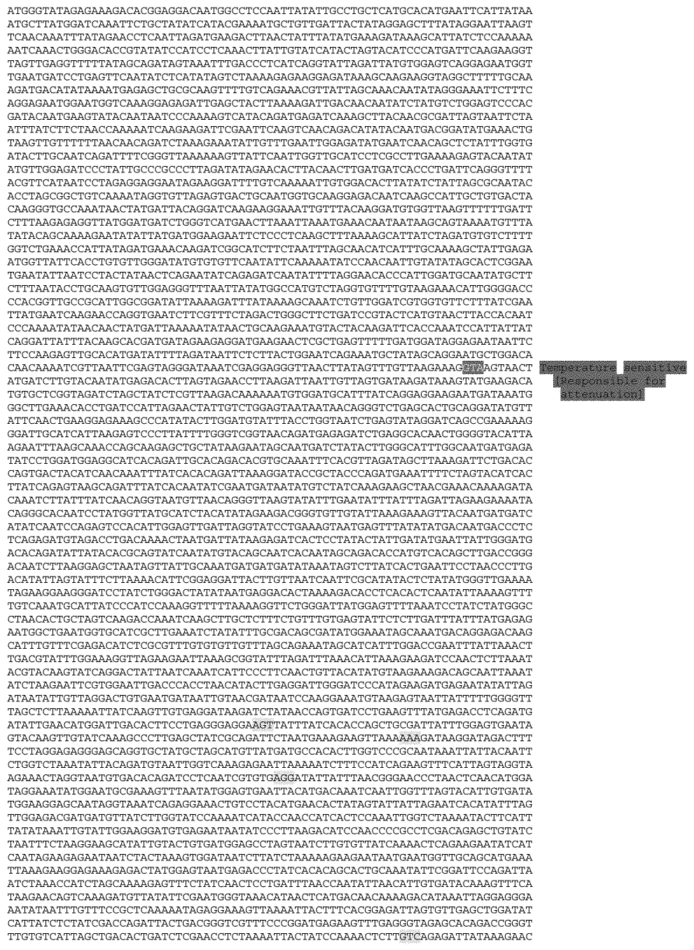

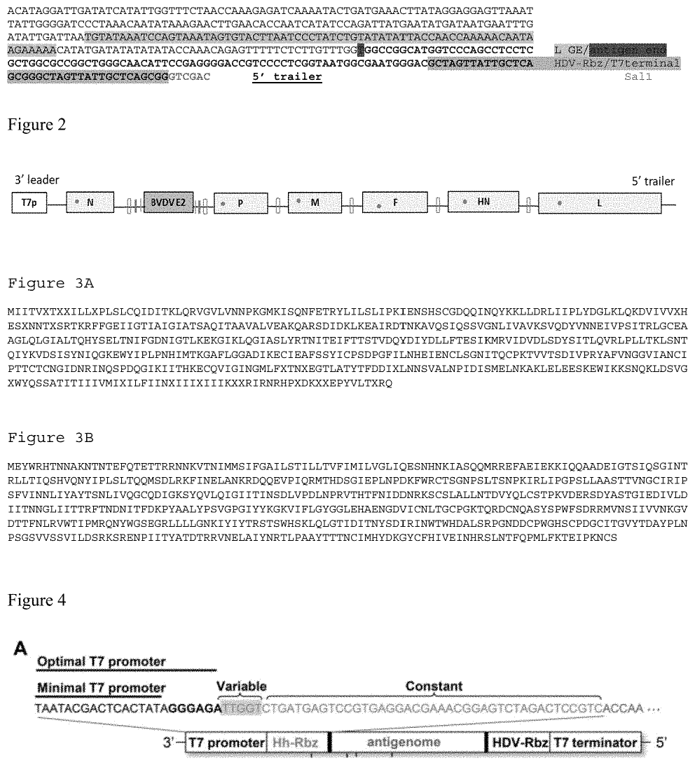

Mutant BPI3V TVMDL16 was therefore designed for the insertion to be placed closer to 3′ end of the genome, immediately downstream of the Nucleoprotein as illustrated in FIG. 2. As can be seen in FIG. 2, which provides the design of the BPI3Vc-E2^{b} backbone, the BVDV E2^{b} transgene is located between N and P, which has been shown to be suitable transgene insertion site for generation of recombinant BPI3V constructs. The green dots indicate location of attenuating mutations based on the current BPI3Va vaccine virus strain [Kansas/15626/84]. Specifically, I 1103 V mutation in the polymerase gene (L) is responsible for temperature sensitive [Ts mutant] attenuation.

With the intention of expressing the insertion sequence on the surface of the virus, the idea will be to mimic the assembly of the BPI3V Fusion protein. Hence a Fusion (F) gene start sequence, transmembrane, cytoplasmic domains flank the insertion sequence. PAM sites for possible exploration with CRISPR and restriction sites are placed as shown above in order to allow insertion of target genes

Optimized T7 Expression Promoter:

Reverse genetics system for rescue of negative stranded RNA Paramyxoviruses from plasmids employs the bacteriophage T7 RNA polymerase. This can be obtained in three ways (i) co-infecting cells with vaccinia virus expressing T7, transfecting cell lines that constitutively co-express T7, or (iii) co-transfecting cells with a plasmid expressing T7 polymerase. Rescue efficiency was demonstrated to be significantly increased by use of a T7 polymerase gene codon optimized for expression in mammalian cells (BSR-T7/5 cells) which also constitutively express T7 polymerase. In this case, the promoter sequence in the vector backbone is also respectively codon optimized in line with the optimized polymerase gene. Additionally, an autocatalytic hammerhead ribozyme sequence (Hh-Rbz) introduced downstream of the Optimal T7 promoter self-cleaves immediately before the start of the antigenome therefore ensuring that the rule of six is adhered to. The variable region at the start of the Hh-Rbz is the reverse complement of the start of the antigenome, while the constant region is fixed. The BPI3Vc vector was modified to have similar Optimal T7 promoter and Hh-Rbz as shown in the figure below. We also obtained the Optimized T7 polymerase gene in pCAGGSS (Plasmid #65974) deposited to Addgene by the authors and as shown in FIG. 4.

The entire modified BPI3Vc vector containing a codon-optimized gene encoding BVDV-Ib E2 mosaic antigen fused in-frame to FLAG tag was synthesized and cloned into pUC-SP (outsourced from Bio Basic, Canada) to generate a construct designated pUCBPI3Vc-E2^{b}(insert sequence). Upon receipt of the synthesized product and conducting QC by restriction digest, the pUCBPI3Vc-E2^{b}(insert sequence) was then used as a template to PCR virus rescue helper genes: i.e. the N gene, P gene, and L gene.

Cloning of Helper Plasmids.

Primers were designed to PCR the N, P, and L genes from the pUCBPI3Vc-E2^{b}(insert sequence) construct. The Optimized T7 promoter region was included in the primer design in order to clone the genes in a suitable cloning vector and be able to increase the expression efficiency in the BSR-T7/5 cells while using the Optimized T7 polymerase gene. Using the same format for the codon optimized T7 expression of the vector, the variable region of each helper plasmid was designed according to its respective reverse complement of the start of its respective antigenome.

BPI3V N Fwd
(SEQ ID NO. 4)
5′ GCGTCGACTAATACGACTCACTATAGGGAGAAACATCTGATGAGTCC
GTGAGGACGAAACGGAGTCTAGACTCCGTCATGTTGAGTCTGTTTGATAC
ATTCAGTGCACGCA 3′
BPI3V N Rev
(SEQ ID NO. 5)
5′ GCAAGCTTTTAGCTACTTCCGAATGCGCTGAACAGGTC 3′
BPI3V P Fwd
(SEQ ID NO. 6)
5′ GCGTCGACTAATACGACTCACTATAGGGAGATCCATCTGATGAGTCC
GTGAGGACGAAACGGAGTCTAGACTCCGTCATGGAAGACAATGTTCAAAA
CAATCAAATCATGG 3′
BPI3V P Rev
(SEQ ID NO. 7)
5′ GCAAGCTTCTATTGGGAGCTAATGTCTTCATTAAACATATCCATCAA
TTCAGATACTTCT 3′
BPI3V L Fwd-1
(SEQ ID NO. 8)
5′ GCCCCGGGTAATACGACTCACTATAGGGAGATCCATCTGATGAGTCC
GTGAGGACGAAACGGAGTCTAGACTCCGTCATGGACACCGAATTCAGCGG
TGGC 3′
BPI3V L Rev
(SEQ ID NO. 9)
5′ GCAAGCTTTTAATCAATATCAAATTCATTATCATATTCATAATCTGG
ATATGATTGGTGT 3′

PCR amplified N, P, and L genes were cloned into pCR4-TOPO vector and QC by restriction digest and sequencing.

Recombinant BPI3Vc-E2^{b}(Insert Sequence) Virus Rescue and Amplification.

Virus Rescue and Amplification

Seed BSR-T7/5 cells at 4×10⁵ per well in a 6-well plate in order to achieve ˜50% confluence on the next day of transfection.

Transfection constructs: Use the following amounts of N, P, and L helper plasmid constructs, and a plasmid encoding T7 polymerase:

5 μg pUCBPI3Vc-E2^{b} (insert sequence) construct

1.5 μg N construct

0.8 μg P construct

0.1 μg L construct

5 μg of T7 polymerase construct

Transfection Reagents:

Set up 1:

5.5 μl PLUS reagent

9 μl Lipofectamine LTX

200 μl Opti-MEM

Set up 2:

2.5 μl PEI per microgram of DNA

200 μl Opti-MEM

Add the transfection reagents (PEI or Lipofectamine/PLUS reagent diluted in 25 μl Optimem) to the plasmid constructs (diluted in 25 μl Optimem) and mix by pipetting gently.

Transfer the constructs/transfection reagents to 150 μl of Optimem.

Incubate at room temperature for 30 min.

Add the transfection mixture gently onto the cells (It is critical that mixture not be agitated before adding to cells, as mixing can disrupt the liposomes at this point).

Incubate at 37° C. for 72 hours (3 days).

At 72 hours post-transfection, harvest the P (0) media and cells and freeze-thaw only the cells (one cycle). Spin down and mix the clean supernatant. Use this to infect fresh MDBK cell monolayer. Stain a portion of the 6 well plate with anti-Flag/E2-specific mAb or sera/anti-BPI3V reference serum to confirm virus assembly.

Infect fresh monolayer of MDBK cells in a T25 flask with the lysate to generate P1 virus stock. Incubate at 37° C. for 5 days.

Stain a portion of the T25 flask as above to confirm virus replication.

Harvest the P (1) virus stock as above and infect fresh monolayer of MDBK cells in a T75 flask.

Incubate at 37° C. for 5 days, harvest P (2) virus and infect a 6 well MDBK plate for 3 days (72 hours) for staining as shown below;

Stain the 6 well plate with:

Anti-Flag antibody—confirm insert sequence expression.

Anti-BPI3V IgG polyclonal antibody—confirm that the virus assembled is BPI3V.

Anti-E2 monoclonal antibody—confirm E2 protein insert sequence expression.

Amplify virus in T7 then T175 flasks and conduct confirmatory QC at each time point QC (by staining as above). Purify virus by sucrose gradient and determine virus titer. Conduct QC of the purified virus and conduct in vivo studies to determine attenuation and vaccine efficacy.

FIG. 5 illustrates an attenuated BPI3Vc-E2^{b} virus expressing the E2^{b} transgene. For this figure, the recombinant BPI3Vc virus expressing the FLAG-tagged E2^{b} transgene was rescued by transfecting BSR-T7/5 cells, which constitutively express the T7 RNA polymerase with the pBPI3Vc-E2^{b} construct in the presence of the pCR4-N, pCR4-P, and pCR4-L helper constructs. Lysate and supernatant from the transfected cells was used to infect MDBK and 72 hrs. post-infection, expression of the FLAG-tagged E2^{b} was evaluated by immunocytometric analysis using anti-FLAG monoclonal antibody. The rescued virus can be scaled up, and tested for attenuation in vitro and in vivo. It can also be used to conduct a pilot immunogenicity and protective efficacy against BPI3V genotype C strains.

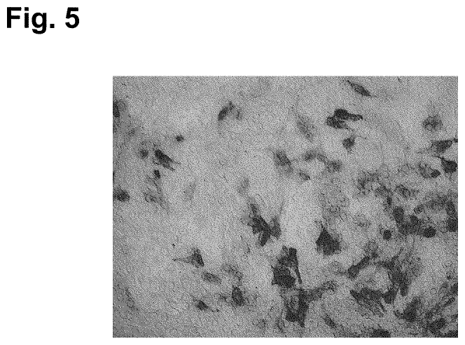

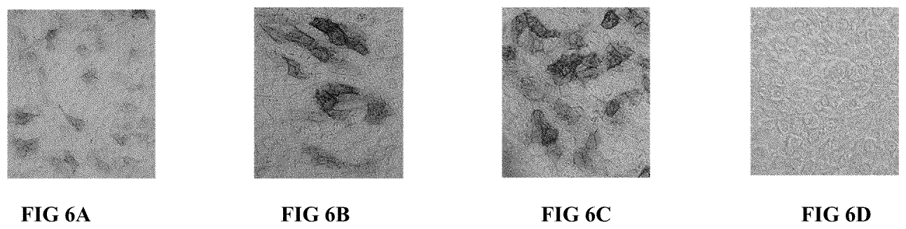

FIGS. 6A-6D are photographs illustrating the surface display of a BVDV E2^{b} transgene on cells infected with BPI3Vc-E2^{b} virus. Rescued recombinant BPI3Vc-E2^{b} virus was used to infect MDBK cells and at 72 hours post-infection, immunocytometric analysis of unfixed cells was used to validate expression of BVDV E2b transgene by BPI3Vc-E2b virus on cell surface using (FIG. 6A) anti-FLAG monoclonal antibody; (FIG. 6B) BPI3V polyclonal antibody (detects expression of BPI3Vc antigens); (FIG. 6C) BVDV Type 1&2 monoclonal antibody (mAb 348) against E2; and (FIG. 6D) uninfected negative control. This is QC data shows that the BVDV E2^{b} transgene is expressed on the surface of cells infected with the BPI3Vc-E2^{b} virus [6A, 6C]. The data [6B] also shows that the rescued virus is strongly recognized by BPI3V reference serum (APHIS 475 BDV 0601).

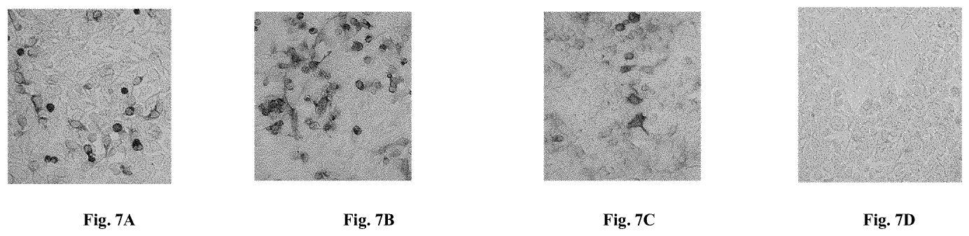

FIGS. 7A-D are photographs illustrating the authenticity of mosaic BPI3V F2-HN2 expressed by plasmid constructs. The expression and authenticity of FLAG-tagged mosaic novel fusion [F2] and HIS-tagged Hemagglutinin-Neuraminidase [HN2] proteins was evaluated by immunocytometric analysis of HEK-293A cells transfected with plasmid constructs and probed with (FIG. 7A) Anti-FLAG monoclonal antibody to detect the FLAG-tagged Fusion protein; (FIG. 7B) Anti-HIS monoclonal antibody to detect the HIS-tagged Hemagglutinin-Neuraminidase protein; (FIG. 7C) BPI3V polyclonal antibody and (FIG. 7D) pCDNA construct negative control. In some forms, the F2-HN2 open-reading frame is separated by a 2A autocleavable motif to allow generation of the individual F2 and HN2 antigens. This data shows expression of the novel mosaic F2HN2 antigens. Antigen expression was validated using anti-tag mAbs and then authenticated using the BPI3V reference serum mentioned above.

### Example 2

Recombinant BPI3Vc viruses that are efficiently expressing the encoded novel F2-HN2, or the E2-NS2-5 mosaic antigens were generated, and showed that they are temperature sensitive.

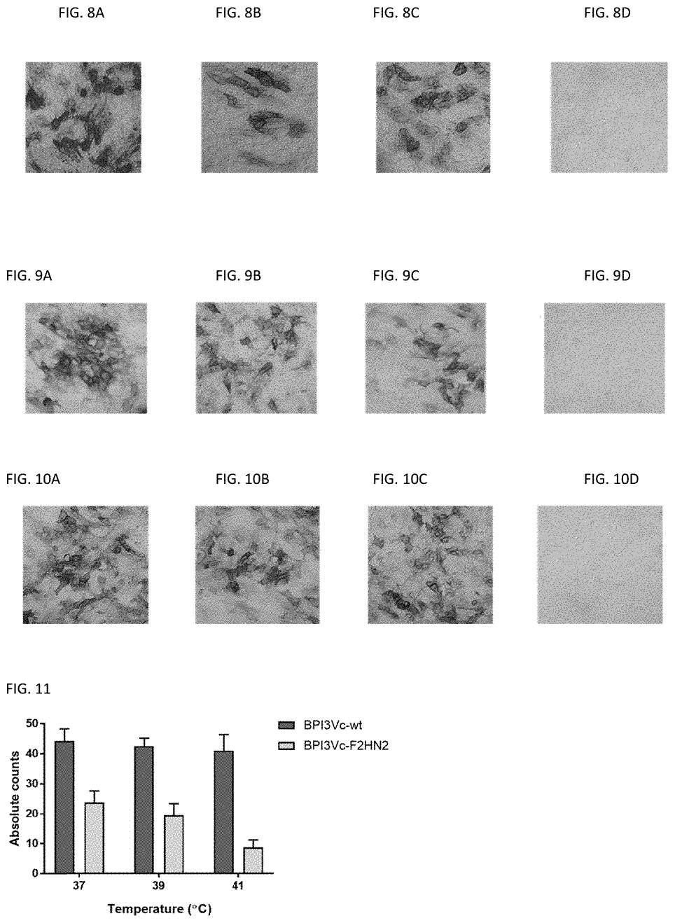

Rescued recombinant BPI3Vc-E2-NS2-3b virus was used to infect MDBK cells and at 72 hours post-infection, immunocytometric analysis was used to validate expression of the transgene using: A) Anti-FLAG mAb (FIG. 8A); B) BPI3V reference serum (FIG. 8B); C) BVDV E2-specific mAb 348 (FIG. 8C); and D) uninfected negative control cells probed with the anti-BPI3V reference serum (FIG. 8D). Staining unfixed cells (FIG. 8C) showed that the E2 antigen is surface displayed. Similar results were obtained for the BPI3Vc virus expressing E2-NS2-3^{a}; and E2-NS2-3². The virus dose used was not normalized.

Rescued recombinant BPI3Vc-NS4-5¹ virus was used to infect MDBK cells and at 72 hours post infection, immunocytometric analysis was used to validate expression of the transgene using: A) Anti-FLAG mAb (FIG. 9A); B) BPI3V polyclonal reference antibody (FIG. 9B); C) Anti-BVDV polyclonal antibody (FIG. 9C); and D) uninfected negative control cells probed with the anti-BPI3V reference serum (FIG. 9D). Similar results were obtained for BPI3Vc-NS4-5² virus.

Rescued recombinant BPI3Vc-F2-HN2 virus was used to infect MDBK cells and at 72 hours post-infection, immunocytometric analysis was used to validate expression of the transgenes: A) FLAG-tagged F2 protein using anti-FLAG mAb [probed unfixed cells to detect surface expression] (FIG. 10A); B) His-tagged HN2 using anti-His mAb (FIG. 10B); C) authenticity of the BPI3Vc, F2, and HN2 proteins was validated using the anti-BPI3V reference serum (FIG. 10C); and D) uninfected negative control cells probed with the anti-BPI3V reference serum (FIG. 10D).

Finally, BPI3Vc-F2HN2 virus was shown to be temperature sensitive [ts]. In a preliminary study, purified IgGs from the anti-BPI3V reference serum were used to enumerate infected cells by immunocytometric analysis of Vero cells infected with either wildtype BPI3Vc [BPI3Vc-wt] virus or the recombinant BPI3Vc-F2HN2 virus incubated for 4 days at the indicated temperature. The results are provided in FIG. 11.

What is claimed is:

1. A vector comprising a Bovine Parainfluenza 3 Virus Type c (BPI3Vc) backbone and at least one antigenic insert sequence from a pathogen other than BPI3V.

2. The vector of claim 1, wherein the vector has at least 80% sequence homology with an isolate selected from the group consisting of KJ647285.1, KJ647287.1, HQ530153.1, KT071671.1, JX969001.1, and LC000638.1.

3. The vector of claim 2, wherein the vector is a mosaic of a combination of these BPI3Vc isolates.

4. The vector of claim 1, wherein the antigenic insert from a pathogen other than BPI3V is from a disease-causing organism.

5. The vector of claim 1, wherein the antigenic insert from a pathogen other than BPI3V is from BVDV.

6. The vector of claim 5, wherein the antigenic insert from BVDV is at least one subunit of BVDV.

7. The vector of claim 5, wherein the antigenic insert from BVDV is selected from the group consisting of F, HN2, NS2, NS3, NS4, NS5, E2, or any combination thereof.

8. The vector of claim 1, wherein the vector has at least 90% sequence homology with SEQ ID NO. 1.

9. The vector of claim 1, wherein the antigenic insert comprises a nucleotide sequence encoding a sequence having at least 90% sequence homology with a sequence selected from the group consisting of 2, 3, 114, 115, 116, and any combination thereof.

10. The vector of claim 1, wherein the antigenic insert comprises a nucleotide sequence encoding a sequence having at least 90% sequence homology with a sequence selected from the group consisting of 2, 3, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 72, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, or any combination thereof.

11. A method of reducing the incidence of or severity of infection associated with bovine parainfluenza comprising the step of administering the vector of claim 1 to an animal in need thereof.

12. The method of claim 11, wherein administration of the vector also reduces the incidence of or severity of infection associated with at least one additional pathogen that is not bovine parainfluenza.

13. The method of claim 11, wherein the at least one additional pathogen includes BVDV.

14. The method of claim 11, wherein the vector is administered systemically.

15. The method of claim 11, wherein the antigenic insert of the vector comprises a nucleotide sequence encoding a sequence having at least 90% sequence homology with a sequence selected from the group consisting of 2, 3, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 72, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, or any combination thereof.

16. The method of claim 11, wherein the vector has at least 80% sequence homology with an isolate selected from the group consisting of KJ647285.1, KJ647287.1, HQ530153.1, KT071671.1, JX969001.1, and LC000638.1.

17. The method of claim 16, wherein the vector is a mosaic of a combination of these BPI3Vc isolates.

18. The method of claim 11, wherein the antigenic insert is from BVDV and is selected from the group consisting of F, HN2, NS2, NS3, NS4, NS5, E2, or any combination thereof.

19. The method of claim 11, wherein the vector has at least 90% sequence homology with SEQ ID NO. 1.

20. The method of claim 11, wherein the antigenic insert comprises a nucleotide sequence encoding a sequence having at least 90% sequence homology with a sequence selected from the group consisting of 2, 3, 114, 115, 116, and any combination thereof.
